SEQUENCE LISTING (33 sequences)

SEQ ID NO: 1 - protein, 393 aa, Yersinia frederiksenii
  fred_WP_087817556.1\optimized translation
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 2 - DNA, 1182 bp, Artificial Sequence
  fred encoding nucleic acid sequence
atgaagagcgcgctgaccttcagccgtcgtattaacccggtttttctggcgttctttgtggttgcgttcctgagcggtattgcgggtgcgctgcaggcgccgaccctgagcctgttcctgagcaccgaggtgaaagttcgtccgctgtgggtgggcctgttctacaccgttaacgcgattgcgggtatcaccgtgagctttgttctggcgaagcgtagcgacctgcgtggcgatcgtcgtaaactgatcctggtgtgctacctgatggcggttggtaactgcctgctgttcgcgtttaaccgtgactatctgaccctgattaccgcgggcgtgctgctggcggcggttgcgaacaccgcgatgccgcagattttcgcgctggcgcgtgagtacgcggataacagcgcgcgtgaagtggttatgtttagcagcattatgcgtgcgcaactgagcctggcgtgggttatcggtccgccgctgagcttcatgctggcgctgaactatggcttcaccctgatgttttgcattgcggcgggtatcttcgtgctgagcgcgctggttgtgtggtttattctgccgagcgtgcagcgtgcggaaccggttatggatgcgccgaccgtggcgcaaggcagcctgttcgcggacaaggatgttctgctgctgtttattgcgagcatgctgatgtggacctgcaacaccatgtacatcattgatatgccgctgtatatcaccgcgagcctgggtctgccggagcgtctggcgggtctgctgatgggcaccgcggcgggtctggaaatcccgattatgctgctggcgggctacagcgtgcgtcgttttggcaagcgtaaaatcatgctgttcgcggtgctggcgggcgttctgttttataccggtctggttctgttcaagtttaaaagcgcgctgatgctgctgcagattttcaacgcgatctttattggtatcgtggcgggtatcggcatgctgtacttccaagacctgatgccgggtcgtgcgggtgcggcgaccaccctgtttaccaacagcattagcaccggcgttatcctggcgggcgtgctgcaaggtgttctgaccgaaacctggggtcacaacagcgtgtatgttatggcgatgattctggcgatcctgagcctgatcatttgcgcgcgtgtgcgtgaagcgtaa

SEQ ID NO: 3 - protein, 342 aa, Neisseria meningitidis
  a2,3-sialyltransferase d29-NST, truncated
MERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 4 - protein, 483 aa, Photobacterium damselae
  a2,6-sialyltransferase, Pd2,6ST
MCNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 5 - DNA, 310 bp, Artificial Sequence
  PflpF promoter element
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 6 - DNA, 310 bp, Artificial Sequence
  PglpF_SD4 promoter element
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 7 - DNA, 310 bp, Artificial Sequence
  Pg.pF_SD7 promoter element
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 8 - DNA, 195 bp, Artificial Sequence
  Plac promoter element
atgcgcaaattgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtacctaaggaggaaacagct

SEQ ID NO: 9 - DNA, 1029 bp, Artificial Sequence
  nucleic acid sequence encoding a2,3-sialyltransferase NST
atggaacgtaacgccgtgagcctgctgaaagaaaaactgtttaacgaagaaggtgaaccggtcaatctgatcttctgctataccattctgcagatgaaagtggcagaacgtattatggctcaacatccgggcgaacgcttttatgtggttctgatgagtgaaaaccgtaacgaaaaatacgattactacttcaaccagatcaaagacaaagcagaacgcgcttatttctttcacctgccgtacggtctgaacaaatcgtttaatttcattccgacgatggcggaactgaaagttaaaagcatgctgctgccgaaagtcaaacgtatctatctggcatccctggaaaaagtgtcaattgcggcctttctgtccacctacccggatgctgaaatcaaaaccttcgatgacggcacgggtaacctgattcaaagctctagttatctgggcgatgaattttctgttaacggtacgatcaaacgtaatttcgcccgcatgatgatcggcgattggtctattgcgaaaacccgcaacgccagtgacgaacattacacgatcttcaaaggcctgaaaaacatcatggatgacggtcgtcgcaaaatgacctacctgccgctgttcgatgccagcgaactgaaaacgggcgacgaaaccggcggtacggttcgtattctgctgggttccccggataaagaaatgaaagaaatctcagaaaaagcagctaaaaacttcaaaatccagtatgtcgcaccgcacccgcgccaaacctacggtctgtcgggtgtgaccacgctgaacagcccgtatgttattgaagattacatcctgcgtgaaattaagaaaaacccgcatacccgctatgaaatctacacgtttttctctggtgcggccctgaccatgaaagattttccgaatgtccacgtgtatgcgctgaaaccggccagtctgccggaagactactggctgaaaccggtgtacgctctgttcacgcaatccggcatcccgattctgacctttgacgataaaaactaa

SEQ ID NO: 10 - DNA, 1452 bp, Artificial Sequence
  Nucleic acid sequence encoding a2,6-sialyltransferase Pd2,ST6
atgtgcaatagcgataacaccagcctgaaagaaaccgttagcagcaatagcgcagatgttgttgaaaccgaaacctatcagctgaccccgattgatgcaccgagcagctttctgagccatagctgggaacagacctgtggcaccccgattctgaatgaaagcgataaacaggcaatcagctttgattttgttgcaccggaactgaaacaggatgagaaatattgctttaccttcaaaggcattaccggtgatcatcgttatattaccaataccaccctgaccgttgttgcaccgaccctggaagtttatattgatcatgcaagcctgccgagcctgcagcagctgattcatattattcaggccaaagatgaatatccgagcaatcagcgttttgttagctggaaacgtgttaccgttgatgcagataatgccaacaaactgaacattcatacctatccgctgaaaggcaataataccagtccggaaatggttgcagcaattgatgaatatgcacagagcaaaaatcgcctgaacatcgaattctataccaataccgcacacgtgtttaataacctgcctccgattattcagccgctgtataataacgagaaagtgaaaattagccatattagcctgtatgatgatggcagcagcgaatatgttagcctgtatcagtggaaagataccccgaacaaaattgaaaccctggaaggtgaagttagcctgctggcaaattatctggcaggcaccagtccggatgcaccgaaaggtatgggtaatcgttataattggcacaaactgtatgacaccgactattactttctgcgcgaagattatctggatgttgaagcaaatctgcatgatctgcgtgattatctgggtagcagcgcaaaacaaatgccgtgggatgaatttgcaaaactgagcgatagccagcagaccctgtttctggatattgttggttttgataaagaacagctgcagcagcagtatagccagagtccgctgccgaattttatctttaccggcaccaccacctgggcaggcggtgaaaccaaagaatactatgcacagcagcaggttaacgtgattaacaatgcaattaatgaaaccagcccgtactatctgggtaaagattatgacctgtttttcaaaggtcatccggctggcggtgttattaatgatattattctgggtagcttcccggatatgattaacattccggcaaaaattagcttcgaggttctgatgatgaccgatatgctgccggataccgttgcaggtattgcaagcagcctgtatttcaccattccggcagataaagtgaactttattgttttcaccagcagcgataccattaccgatcgtgaagaagcactgaaaagtccgctggttcaggttatgctgaccctgggtattgttaaagaaaaagatgttctgttctgggcctaa

SEQ ID NO: 11 - protein, 346 aa, Campylobacter jejuni
  Sialic acid synthase, NeuB, GenBank ID AAK91726.1
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 12 - DNA, 1041 bp, Campylobacter jejuni
  nucleic acid sequence encoding sialic acid synthase, NeuB
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtga

SEQ ID NO: 13 - protein, 372 aa, Campylobacter jejuni
  N-acetylglucosamine-6-phosphate 2-epimerase, NeuC
VKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 14 - DNA, 1119 bp, Campylobacter jejuni
  Nucleic acid sequence encoding N-acetylglucosamine-6-phosphate 2-epimerase, NeuC
gtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatga

SEQ ID NO: 15 - protein, 221 aa, Campylobacter jejuni
  CMP-Neu5Ac synthetase, NeuA
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 16 - DNA, 666 bp, Campylobacter jejuni
  nucleic acid sequence encoding CMP-Neu5Ac synthetase, neuA
atgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 17 - DNA, 2818 bp, Campylobacter jejuni
  neuCBA gene cluster
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 18 - protein, 297 aa, Escherichia coli
  carboxylating nicotinate-nucleotide diphosphorylase, nadC
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 19 - DNA, 894 bp, Escherichia coli
  Nucleic acid sequence encoding carboxylating nicotinate-nucleotide diphosphorylase, nadC
atgccgcctcgccgctataaccctgacacccgacgtgacgagctgctggaacgcattaatctcgatatccccggcgcggtggcccaggcgctgcgggaagatttaggcggaacagtcgatgccaacaatgatattacggcaaaacttttaccggaaaattctcgctctcatgccacggtgatcacccgcgagaatggcgtcttttgcggcaaacgctgggttgaagaggtgtttattcaactggcaggcgacgatgtcaccataatctggcatgtggatgacggcgatgtcatcaatgccaatcaatccttgttcgaacttgaaggcccatcccgcgtgctgttaacgggcgaacgcactgcgcttaattttgtgcaaaccctttcaggagttgccagtaaggtacgccactatgtcgaattgctggaaggcaccaacacgcagttgttggatacgcgcaaaaccttacccggcctgcgttcagctctgaaatacgcggtactttgcggcggcggagcgaatcaccgtctggggctttctgatgccttcctgatcaaagaaaaccatattattgcctccggctcagtgcgccaggcggtcgaaaaagcgtcctggctgcacccggatgcgccagtagaagtcgaagtagagaatctggaagaacttgatgaagccctgaaagcaggagccgatatcatcatgctggataacttcgaaacagaacagatgcgcgaagccgtcaaacgcaccaacggcaaggcgctactggaagtgtctggcaacgtcactgacaaaacactgcgtgaatttgccgaaacgggcgtggactttatctccgtcggtgcgctaactaaacacgtacaagcactcgacctttcaatgcgttttcgctaa

SEQ ID NO: 20 - DNA, 24 bp, Artificial Sequence
  Oligo O48, galK.for
cccagcgagacctgaccgcagaac

SEQ ID NO: 21 - DNA, 24 bp, Artificial Sequence
  Oligo O49, galK.rev
ccccagtccatcagcgtgactacc

SEQ ID NO: 22 - DNA, 34 bp, Artificial Sequence
  Oligo O40 Backbone.for
  misc_RNA 9
attaacccuccaggcatcaaataaaacgaaaggc

SEQ ID NO: 23 - DNA, 32 bp, Artificial Sequence
  Oligo O79, Backbone.rev
  misc_RNA 10
atttgcgcaucaccaatcaaattcacgcggcc

SEQ ID NO: 24 - DNA, 32 bp, Artificial Sequence
  Oligo O261, PglpF.for
  misc_RNA 10
atgcgcaaaugcggcacgccttgcagattacg

SEQ ID NO: 25 - DNA, 36 bp, Artificial Sequence
  Oligo O262, PglpF.rev
  misc_RNA 8
agctgttucctccttggttaatgtttgttgtatgcg

SEQ ID NO: 26 - DNA, 36 bp, Artificial Sequence
  Oligo O459, PglpF_SD4
  misc_RNA 8
agctgttucctagttggttaatgtttgttgtatgcg

SEQ ID NO: 27 - DNA, 36 bp, Artificial Sequence
  Oligo O462, PglpF_SD7
  misc_RNA 8
agctgttutgctcttggttaatgtttgttgtatgcg

SEQ ID NO: 28 - DNA, 31 bp, Artificial Sequence
  Oligo KABY733, fred.for
  misc_RNA 8
aaacagcuatgaagagcgcgctgaccttcag

SEQ ID NO: 29 - DNA, 29 bp, Artificial Sequence
  Oligo KABY 734, fred.rev
  misc_RNA 9
agggttaauttacgcttcacgcacacgcg

SEQ ID NO: 30 - DNA, 32 bp, Artificial Sequence
  Oligo O68, Plac.for
  misc_RNA 10
atgcgcaaautgtgagttagctcactcattag

SEQ ID NO: 31 - DNA, 36 bp, Artificial Sequence
  Oligo O113, Plac.rev
  misc_RNA 8
agctgttucctccttaggtacccagcttttgttccc

SEQ ID NO: 32 - DNA, 32 bp, Artificial Sequence
  Oligo KABY721, YberC0001_9420.for
  misc_RNA 8
aaacagcuatgaagagcgcgctgacctttagc

SEQ ID NO: 33 - DNA, 29 bp, Artificial Sequence
  Oligo KABY722, YberC0001_9420.rev
  misc_RNA 9
agggttaauttacgcctcacgcacacgcg

(19) United States
(12) Patent Application Publication
(10) Pub. No.: US 2024/0102063 A1
(43) Pub. Date: Mar. 28, 2024

(54) NEW MAJOR FACILITATOR SUPERFAMILY (MFS) PROTEIN (FRED) IN PRODUCTION OF SIALYLATED HMOS

(71) Applicant: GLYCOM A/S, Hørsholm (DK)

(72) Inventors: Margit PEDERSEN, Hørsholm (DK); Isotta D'ARRIGO, Hørsholm (DK); Katrine BYCH KAMPMANN, Hørsholm (DK); Manos PAPADAKIS, Brønshøj (DK)

(21) Appl. No.: 18/262,099

(22) PCT Filed: Jan. 21, 2022
(86) PCT No.: PCT/EP2022/051295

§ 371 Date: Jul. 19, 2023

(30) Foreign Application Priority Data
Jan. 22, 2021 (WO) PCT/EP2021/051479
Jul. 13, 2021 (EP) 21185379.1

(51) Int. Cl. C12P 19/04; C07K 14/24; C12N 9/10; C12N 15/52; C12P 19/18

(52) U.S. Cl. CPC C12P 19/04; C07K 14/24; C12N 9/1081; C12N 15/52; C12P 19/18; C12Y 204/99001; C12Y 204/99004

(57) ABSTRACT

The present invention relates to the field of recombinant production of biological molecules in genetically modified cells. More particularly, it relates to a method for recombinant production of sialylated human milk oligosaccharides (HMO) using a genetically modified cell expressing a protein of the major facilitator superfamily (MFS), the protein expressed being Fred.

### FIELD

The present invention relates to the field of recombinant production of biological molecules in genetically modified cells. More particularly, it relates to a method for recombinant production of sialylated human milk oligosaccharides (HMOs) using a genetically modified cell expressing a protein of the major facilitator superfamily (MFS), the protein expressed being Fred.

### BACKGROUND

Human milk oligosaccharides (HMOs) are non-digestible carbohydrates and constitute the third largest component of mother's milk. No other mammal has a similar concentration or complexity of non-digestible oligosaccharides compared to human mother's milk. To date, more than 200 HMO's have been identified (see XI Chen, Chapter 4 of Advances in Carbohydrate Chemistry and Biochemistry, 2015, Volume 72 and Urashima et al.: Milk Oligosaccharides, Nova Biomedical Books, New York, 2011, ISBN: 978-1-61122-831-1).

HMOs have become of great interest in the last decade, due to the discovery of their important functionality in human development. Besides their prebiotic properties, HMOs have been linked to additional positive effects, which expands their field of application (Kunz C. et al., (2014) Food Oligosaccharides: Production, Analysis and Bioactivity, 1st Edition, p 5-20, Eds. Moreno J. and Luz Sanz M., John Wiley & Sons, Ltd). The health benefits of HMOs have enabled their approval for use in foods, such as infant formulas and foods, and for consumer health products.

HMOs can be synthesised chemically; this however poses a challenge in terms of producing large-scale quantities. To overcome the challenges associated with the chemical synthesis of HMOs, several enzymatic methods and fermentative approaches have been developed. Fermentation based processes have been developed for several HMOs, such as 2′-fucosyllactose, 3-fucosyllactose, lacto-N-tetraose, lacto-N-neotetraose, 3′-sialyllactose and 6′-sialyllactose. Fermentation based processes typically utilize genetically modified bacterial strains, such as recombinant Escherichia coli (E. coli) (for review see Bych et al, Current Opinion in Biotechnology 2019, 56: 130-137).

Recent developments in biotechnological production of HMOs have made it possible to overcome certain inherent limitations of bacterial expression systems. For example, HMO-producing bacterial cells may be genetically modified to increase the limited intracellular pool of nucleotide sugars in the bacteria (WO2012/112777), to improve activity of enzymes involved in the HMO production (WO2016/040531), or to facilitate the secretion of synthesized HMOs into the extracellular media (WO2010/142305, WO2017/042382). Further, expression of genes of interest in recombinant cells may be regulated by using particular promoters or other gene expression regulators, for example as described in WO2019/123324.

The use of sugar efflux transporters has been described in WO2010/142305 and WO2017/042382 and has the advantage of reducing the metabolic burden inflicted on the cell by producing high levels of HMOs by allowing the HMOs to leave the cell during production. This approach attracts growing attention in recombinant HMO-producing cell-engineering, e.g., recently there have been described several new sugar transporter genes encoding proteins and fermentation processes that can facilitate efflux of a recombinantly produced 2′-fucosyllactose (2′-FL), the most abundant HMO of human milk (WO2018/077892, US2019/00323053, US2019/00323052).

Production of sialylated HMOs has been described in WO2007/101862, describing the modifications needed to produce for example 3′-SL from a microorganism. The production of sialylated HMOs results in acidification of the cell due to the acidity of the sialyl moiety, which in turn leads to a less favourable over-all production outcome, due to the physiological impact of cellular acidification. Thus, an enhanced export of in particular the sialylated HMOs from the cell would have a physiological benefit for the cell, while also simplifying the downstream harvest and purification of the sialylated HMO, overall having a great benefit in a large-scale production of sialylated HMOs.

However, at present, there is no algorithm that is able to pinpoint the right transporter protein capable of efflux of different recombinantly produced HMO structures among numerous bacterial proteins with predicted transporter function in multiple protein databases, e.g., UniProt, since the structure-function relationship defining substrate specificity of sugar transporters is still not well-studied and remains highly unpredictable.

The present disclosure for the first time shows the use of a specific heterologous sugar efflux transporter protein in a sialylated HMO producing strain for enhancing the amount of product produced alongside other favourable production benefits.

### SUMMARY

Recently, the inventors of the present application have identified several transporters in the major facilitator superfamily (MFS) capable of transporting other HMOs, such as 3-FL, LNT, LNT-II, LNnT and LNFP-I (WO2021/148615 and WO2021/148614 and WO2021/148611 and WO2021/110610 and PCT/EP2021/0514662 and WO2021/148620 and WO2021/148618). The present disclosure for the first time shows the use of the heterologous sugar efflux transporter protein Fred in a sialylated HMO producing strain for favourable production benefits.

The present disclosure shows that overexpression in sialylated HMO producing strains of the heterologous gene fred, which encodes the Fred protein from the Major facilitator superfamily (MFS), increases the amount of HMO exported out of the cellsm without affecting the over all production of the HMO. Identification of new efficient sugar efflux transporter proteins having specificity for different recombinantly produced HMOs and development of recombinant cells expressing said protein are advantageous for large-scale industrial HMO manufacturing.

The produced HMOs can comprise one or more sialyl moieties. In that regard the produced HMOs may be selected from the group consisting of 3′-SL (3′-Sialyllactose), 6′-SL (6′-Sialyllactose), LSTc (sialyllacto-N-neotetraose c), LSTa (sialyllacto-N-tetraose a), LSTb (sialyllacto-N-tetraose b), 3′-S,3-FL (sialyl-3-fucosyllactose) and DS-LNT (disialyllacto-N-tetraose). In particular, 3′-SL and 6′-SL are of interest since these are the most abundant sialylated HMOs.

The present invention thus relates to a genetically modified cell capable of producing one or more sialylated Human Milk Oligosaccharides (HMOs), wherein said genetically modified cell comprises a heterologous nucleic acid sequence encoding a major facilitator superfamily (MFS) polypeptide of SEQ ID NO: 1, or a functional homologue thereof having an amino acid sequence which is more than 70% identical to SEQ ID NO: 1, such as at least 80%, such as at least 90%, such as at least 95% such as at least 99% or such as at least 99.7% identical to SEQ ID NO: 1.

Furthermore, the genetically modified cell expresses a sialyl-transferase. In particular a sialyl-transferase is selected from the group consisting of α-2,3- sialyltransferases and α-2,6- sialyltransferases, such as the sialyltransferases of table 2.

In one embodiment, the Fred expressing genetically modified cell may further be modified to heterologously express a α-2,3-sialyl-transferase of SEQ ID NO: 3 and/or a functional homologue thereof having an amino acid sequence which is at least 70% identical to SEQ ID NO: 3, such as at least 80%, such as at least 90%, such as at least 95% such as at least 99% or such as at least 99%. In addition, the genetically modified cell contains a biosynthetic pathway for making a sialate sugar nucleotide.

In one embodiment, the Fred expressing genetically modified cell may further be modified to heterologously express a α-2,6-sialyl-transferase of SEQ ID NO: 4 and/or a functional homologue thereof having an amino acid sequence which is at least 70% identical to SEQ ID NO: 4, such as at least 80%, such as at least 90%, such as at least 95% such as at least 99% or such as at least 99%. In addition, the genetically modified cell contains a biosynthetic pathway for making a sialate sugar nucleotide.

In one embodiment, the Fred expressing genetically modified cell may further be modified to heterologously express a α-2,3-sialyl-transferase of SEQ ID NO: 3 and a α-2,6-sialyl-transferase of SEQ ID NO: 4 and/or a functional homologue of SEQ ID NO: 3 or 4, having an amino acid sequence which is at least 70% identical to SEQ ID NO: 3 or 4, such as at least 80%, such as at least 90%, such as at least 95% such as at least 99% or such as at least 99%. In addition, the genetically modified cell contains a biosynthetic pathway for making a sialate sugar nucleotide.

The genetically modified cell according to the present invention can further comprise a nucleic acid sequence comprising a regulatory element for the regulation of the expression of the heterologous nucleic acid sequence(s). The regulatory element can regulate the expression of a nucleic acid encoding a polypeptide of interest and can be selected from the group consisting of PglpF, PglpF_SD4, and PglpF_SD7.

In one embodiment of the invention said regulatory element regulates the expression of the MFS polypeptide shown in SEQ ID NO: 1, or a functional homologue thereof having an amino acid sequence which is more than 70% identical to SEQ ID NO: 1, such as at least 80, such as at least 90%, such as at least 95% such as at least 99% or such as at least 99.7% identical to SEQ ID NO: 1.

In a preferred aspect, said regulatory element regulates the expression of the MFS polypeptide shown in SEQ ID NO: 1, or a functional homologue thereof having an amino acid sequence which is more than 95.4%, such as more than 99.7%, such as 100% identical to SEQ ID NO: 1. The present invention shows that use of HMO producing recombinant cells that express Fred protein results in very distinct improvements of the HMO manufacturing process related both to fermentation and purification of the HMOs. The recombinant cells and methods for HMO production disclosed herein provide both higher secretion of HMOs from the cell to the supernatant, lower by-product formation or by-product-to-product ratio and facilitated recovery of the HMOs during downstream processing of the fermentation broth.

Expression of a DNA sequence encoding Fred in different HMO producing cells is found to be associated with accumulation of some particular HMOs in the extracellular media and other HMOs inside of the producing cells, and in an increase in total production of the HMOs (see WO2021/148620). Surprisingly, an increase in the efflux of the produced HMOs is found to be characteristic for HMOs that consist of either tri or tetra units of monosaccharides, i.e. HMOs that are trisaccharides or tetrasaccharides, e. g, 2′-fucosyllactose (2′-FL), 3-fucosyllactose (3-FL), 3-sialyllactose (3′-SL), 6′-sialyllactose (6′-SL), lacto-N-triose 2, (LNT-2), lacto-N-neotetraose (LNnT) or lacto-N-tetraose (LNT), especially for 2′-fucosyllactose (2′-FL), 3-fucosyllactose (3-FL), or lacto-N-tetraose (LNT) as seen from the examples herein, but not for larger oligosaccharide structures, like pentasaccharides or hexasaccharides, which accumulate inside of the producing cells.

In one aspect, the HMO produced by said cell is a sialylated HMO, such as but not limited to, one or more Human Milk Oligosaccharides selected from the group consisting of 3′-SL and 6′-SL. In a preferred embodiment, the sialylated HMO is 3′-SL.

More surprisingly, it was found that the HMO, 3′-SL, in the corresponding HMO producing cells expressing the fred gene, is almost totally 3′-SL and almost exclusively found in the fermentation media.

The invention also relates to a nucleic acid construct comprising a nucleic acid sequence encoding a polypeptide of the Major facilitator superfamily (MFS), wherein the nucleic acid sequence encoding the Major facilitator superfamily (MFS) polypeptide has at least 70%, more preferably at least 80%, more preferably at least 90%, more preferably at least 95% and even more preferably at least 99% sequence identity to SEQ ID NO: 2, also the invention relates to a genetically modified cell comprising the nucleic acid construct, which is Escherichia coli.

Furthermore, the invention provides a method for the biosynthetic production of one or more sialylated Human Milk Oligosaccharides (HMOs), the method comprising the steps of:
(i) providing a genetically modified cell according to the present invention;
(ii) culturing the genetically modified cell according to (i) in a suitable cell culture medium to express said polypeptide capable of efflux sugar transportation and produce one or more sialylated Human Milk Oligosaccharides (HMOs) and;
(iii) harvesting one or more sialylated HMOs produced in step (ii).

The invention also relates to the use of a genetically modified cell or a nucleic acid construct comprising a heterologous nucleic acid sequence encoding a Major facilitator superfamily (MFS) polypeptide, said nucleic acid sequence having at least 70% sequence identity to SEQ ID NO: 2, for the biosynthetic production of one or more sialylated Human Milk Oligosaccharides (HMOs).

In a preferred embodiment, the invention also relates to the use of a genetically modified cell or a nucleic acid construct comprising (i) one or more nucleic acid sequence(s) encoding an MFS polypeptide according to SEQ ID NO: 1, or a functional homologue thereof, having more than 70%, 80%, 94.5% or 99.7% sequence identity to SEQ ID NO: 1, wherein the nucleic acid sequence encoding the MFS polypeptide has at least 70%, such as at least 80% sequence identity to SEQ ID NO: 2, (ii) one or more heterologous nucleic acid sequence(s) encoding one or more polypeptides with sialyl-transferase capabilities, and one or more a nucleic acid sequence comprising a regulatory element, that regulates the expression of any one or more of the nucleic acid sequence(s) of point (i) and/or (ii).

As mentioned above, during the culturing of genetically modified cells capable of producing one or more sialylated HMOs, which cells comprise a nucleic acid sequence encoding a Fred transporter protein, it has surprisingly been found that the corresponding one or more sialylated HMOs are produced in high yields, while by-product and biomass formation is reduced. This facilitates improved recovery of the HMOs during downstream processes, e.g., the overall recovery and purification procedure may comprise less steps and overall time of purification may be shortened.

In particular, the effects of improved product recovery makes the present invention superior to the disclosures of the prior art.

Other aspects and advantageous features of the present invention are described in detail and illustrated by non-limiting working examples below.

### FIGURES

FIG. 1 shows the relative 3′-SL production of a modified E. coli strain (strain 2) with and (strain 1) without integration of the fred gene of SEQ ID NO: 2, encoding for the expression of the Fred MFS transporter protein of SEQ ID NO: 1, in strains 2 and 1, respectively. Data is obtained from a deep-well feed batch assay

FIG. 2 shows the relative distribution of 3′-SL inside and outside the cells of a modified E. coli strain with (strain 2) and without (strain 1) the integration of the fred gene of SEQ ID NO: 2, encoding for the expression of the Fred MFS transporter protein of SEQ ID NO: 1. Data is obtained from a deep-well feed batch assay.

FIG. 3 shows the time profiles of 3′-SL distribution between the pellet and supernatant fraction of the fermentation broth in cells of a modified E. coli strain with (strain 2, batch GDF21173 and GDF21177) and without (strain 1, batch GDF21170 and GDF21174) the integration of the fred gene of SEQ ID NO: 2, encoding for the expression of the Fred MFS transporter protein of SEQ ID NO: 1.

FIG. 4 shows the time profiles of bio wet mass (BWM) development throughout the fermentation in cells of a modified E. coli strain with (strain 2 batch GDF21173 and GDF21177) and without (strain 1 batch GDF21170 and GDF21174) the integration of the fred gene of SEQ ID NO: 2, encoding for the expression of the Fred MFS transporter protein of SEQ ID NO: 1.

FIG. 5 shows the time profiles of 3′-SL distribution between the pellet and supernatant fraction of the fermentation broth in cells of a modified E. coli strain with (strain 4) and without (strain 3) the integration of the fred gene of SEQ ID NO: 2, encoding for the expression of the Fred MFS transporter protein of SEQ ID NO: 1.

FIG. 6 shows the time profiles of bio wet mass (BWM) development throughout the fermentation in cells of a modified E. coli strain with (strain 4) and without (strain 3) the integration of the fred gene according to SEQ ID NO: 2, encoding for the expression of the Fred MFS transporter protein according to SEQ ID NO: 1. Strain 3 and Strain 4 have neuBCA genes integrated and overexpressed from the genome.

FIG. 7 Shows the relative production of 3′-SL (A) in cells expressing the alternative MFS transporter, YberC (strain 5), as well as the distribution between pellet and supernatant (B), compared to cells without transporter (strain 1). Data is obtained from a deep-well feed batch assay.

### DETAILED DESCRIPTION

In the following, embodiments of the invention will be described in further detail. Each specific variation of the features can be applied to other embodiments of the invention unless specifically stated otherwise.

Generally, all terms used herein are to be interpreted according to their ordinary meaning in the technical field, and applicable to all aspects and embodiments of the invention, unless explicitly defined or stated otherwise.

All references to “a/an/the [cell, sequence, gene, transporter, step, etc]” are to be interpreted openly as referring to at least one instance of said cell, sequence, gene, transporter, step, etc., unless explicitly stated otherwise.

The steps of any method disclosed herein do not have to be performed in the exact order disclosed, unless explicitly stated.

The present invention in general relates to a genetically modified cell for efficient production of oligosaccharides and use of said genetically modified cell in a method of producing the oligosaccharides. In particular, the present invention relates to a genetically modified cell enabled to synthesize an oligosaccharide, preferably a heterologous oligosaccharide, in particular a human milk oligosaccharide (HMO).

Accordingly, a genetically modified cell of the invention is a host cell that is modified to express a set of recombinant nucleic acids that are necessary for synthesis of one or more HMOs by the cells (which enable the host cell to synthesize one or more HMOs), such as genes encoding one or more enzymes with glycosyltransferase activity as described below. The oligosaccharide producing recombinant cell of the invention is further modified to comprise a heterologous recombinant nucleic acid sequence, preferably, a DNA sequence, encoding a putative MFS (major facilitator superfamily) transporter protein, originating from the bacterium Yersinia frederiksenii.

Specifically, the invention relates to a genetically modified cell optimized for the production of one or more particular oligosaccharides, in particular one or more particular HMOs, comprising a recombinant nucleic acid encoding a Fred protein.

A nucleic acid sequence encoding a Fred protein having the nucleic acid sequence of SEQ ID NO: 2 is herein identified as “Fred coding nucleic acid/DNA” or “fred gene” or “fred”.

The MFS transporter protein identified herein as “Fred protein” or “Fred transporter” or “Fred MFS (major facilitator superfamily) transporter protein” or “Fred”, interchangeably, has the amino acid sequence of SEQ ID NO: 1; The amino acid sequence identified herein as SEQ ID NO: 1 is an amino acid sequence that has 100% identity with the amino acid sequence having the GenBank accession ID WP_087817556.1.

Accordingly, one aspect of the invention relates to a genetically modified cell capable of producing one or more Human Milk Oligosaccharides (HMO(s)), wherein said genetically modified cell comprises a heterologous nucleic acid sequence encoding a polypeptide capable of sugar transportation, said nucleic acid sequence having at least 70%, more preferably at least 80%, more preferably at least 90%, more preferably at least 95% and even more preferably at least 99% sequence identity to SEQ ID NO: 2.

Accordingly, one aspect of the invention relates to a genetically modified cell capable of producing one or more sialylated Human Milk Oligosaccharides (HMOs), wherein said genetically modified cell comprises a heterologous nucleic acid sequence encoding a polypeptide capable of sugar transportation, said nucleic acid sequence having at least 70%, more preferably at least 80%, more preferably at least 90%, more preferably at least 95% and even more preferably at least 99% sequence identity to SEQ ID NO: 2.

Further, the invention relates to a genetically modified cell optimized for the production of one or more particular oligosaccharides, in particular one or more particular HMOs, comprising a recombinant nucleic acid encoding a protein having more than 95.4%, such as at least 95.5% sequence identity, preferably at least 96%, more preferably at least 97%, more preferably at least 98%, and even more preferably at least 99% sequence identity to the amino acid sequence of SEQ ID NO: 1.

Moreover, in a preferred aspect, the invention relates to a genetically modified cell capable of producing one or more sialylated Human Milk Oligosaccharides (HMOs), comprising a recombinant nucleic acid encoding a protein having more than 70%, such as at least 80% sequence identity, preferably at least 95%, more preferably at least 98%, more preferably at least 99%, and even more preferably at least 99.9% sequence identity to the amino acid sequence of SEQ ID NO: 1.

The presence of the Fred transporter in the genetically modified HMO producing cell results in an increased export of the HMO's, in particular the sialylated HMO, such that a larger fraction of the HMO is in the supernatant of the culture broth as compared to the same cell without the Fred transporter. Preferably at least 80% of the sialylated HMO, such as 3′-SL, 6′-SL, FSL, DS-LNT, LSTc, LSTa and LSTb, is exported out of the cell. In particular at least 80% of 3′-SL or 6′-SL is exported out of the cell. Preferably, the over all production of the HMO, such as 3′-SL or 6′-SL, is not significantly affected by the presence of the Fred transporter.

By the term “functional homolog” in the present context is meant a protein that has an amino acid sequence that is more than 70%, such as 71%-99,9%, such as 95.4%, such as 95.5%-99.7% or 100% identical to SEQ ID NO: 1 and has a function that is beneficial to achieve at least one advantageous effect of the invention, e.g., an increased recovery of the produced HMO(s), HMO production efficiency and/or viability of an HMO producing cell.

By the term “Major Facilitator Superfamily (MFS)” is meant a large and exceptionally diverse family of the secondary active transporter class, which is responsible for transporting a range of different substrates, including sugars, drugs, hydrophobic molecules, peptides, organic ions, etc. The specificity of sugar transporter proteins is highly unpredictable and the identification of novel transporter protein with specificity towards for example oligosaccharides requires unburden laboratory experimentation (for more details see review by Reddy V. S. et al., (2012), FEBS J. 279(11): 2022-2035). The term “MFS transporter” means in the present context a protein that facilitates transport of an oligosaccharide, preferably, an HMO, through the cell membrane, preferably transport of an HMO/oligosaccharide synthesized by the genetically modified cell from the cell cytosol to the cell medium, preferably an HMO/oligosaccharide comprising three or four sugar units, e.g., 2′-FL, 3-FL, LNT-2, LNT, LNnT, 3′-SL or 6′-SL. Additionally, or alternatively, the MFS transporter may also facilitate efflux of molecules that are not considered HMO or oligosaccharides according to the present invention, such as lactose, glucose, cell metabolites or toxins.

The term “Fred” is used to describe one member of the class of MFS transporters. The amino acid sequence identified herein as SEQ ID NO: 1 is the amino acid sequence that is 100% identical to the amino acid sequence having the GenBank accession ID WP_087817556.1. The MFS transporter protein having the amino acid sequence of SEQ ID NO: 1 is identified herein as “Fred protein” or “Fred transporter” or “Fred MFS (major facilitator superfamily) transporter protein” or “Fred”, interchangeably. A nucleic acid sequence encoding Fred protein is identified herein as SEQ ID NO: 2 “Fred coding nucleic acid/DNA” or “fred gene” or “fred”.

The term “sialyl-transferase” as used herein refers to a protein or polypeptide which is capable of transferring sialic acid from an activated donor molecule to an oligosaccharide acceptor forming a glycosidic bond. Sialyl-transferases are grouped in Cazy family 29. One type of sialyl-transferases can add sialic acid with an alpha-2,3 linkage to galactose, while another type of sialyltransferases add sialic acid with an alpha-2,6 linkage to galactose or N-acetylgalactosamine. The activated donor molecule is preferably CMP-sialate or CMP-Neu5Ac.

The term “sequence identity of [a certain] ° %,” in the context of two or more nucleic acid or amino acid sequences means that the two or more sequences have nucleotides or amino acid residues in common in the given percent when compared and aligned for maximum correspondence over a comparison window or designated sequences of nucleic acids or amino acids (i.e., the sequences have at least 90 percent (%) identity). Percent identity of nucleic acid or amino acid sequences can be measured using a BLAST 2.0 sequence comparison algorithm with default parameters, or by manual alignment and visual inspection (see e.g., http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence identity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the invention. Software for performing BLAST analyses is publicly available through the National Center for Biotechnology Information (http://www.ncbi.nlm.nih.gov/). Examples of sequence alignment algorithms are CLUSTAL Omega (http://www.ebi.ac.ukfTools/msa/clustalo/), EMBOSS Needle (http://www.ebi.ac.uk/Tools/psa/emboss_needle/), MAFFT (http://mafft.cbrc.jp/alignment/server/) or MUSCLE (http://www.ebi.ac.uk/Tools/msa/muscle/).

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment−Total Number of Gaps in Alignment).

In the context of the invention, the term “oligosaccharide” means a saccharide polymer containing a number of monosaccharide units. In some embodiments, preferred oligosaccharides are saccharide polymers consisting of three or four monosaccharide units, i.e., trisaccharides or tetrasaccharides. Preferable oligosaccharides of the invention are human milk oligosaccharides (HMOs).

### HMO

The term “human milk oligosaccharide” or “HMO” in the present context means a complex carbohydrate found in human breast milk (for reference, see Urashima et al.: Milk Oligosaccharides. Nova Science Publisher (2011); or Chen, Adv. Carbohydr. Chem. Biochem. 72, 113 (2015)). The HMOs have a core structure comprising a lactose unit at the reducing end that can be elongated by one or more β-N-acetyl-lactosaminyl and/or one or more β-lacto-N-biosyl units, and this core structure can be substituted by an α-L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH).

Examples of acidic and sialylated HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT). In the context of the present invention lactose is not regarded as an HMO species.

In a one preferred aspect of the invention, trisaccharide HMOs are preferred, in particular trisaccharide's selected from 3′-SL and 6′-SL, such as in particular 3′-SL.

In another preferred aspect of the invention, tetrasaccharide HMOs are preferred, in particular tetrasaccharides such as FSL.

### 2′-FL

2′-Fucosyllactose (2′-FL or 2′O-fucosyllactose) is a trisaccharide, more precisely, fucosylated, neutral trisaccharide composed of L-fucose, D-galactose, and D-glucose units (Fucα1-2Galβ1-4Glc). It is the most prevalent human milk oligosaccharide (HMO) naturally present in human breast milk, making up about 30% of all of HMOs. In a genetically modified cell or in an enzymatic reaction, 2′-FL is produced primarily by an α1,2-fucosyltransferase enzymatic reaction with lactose and a fucosyl doner.

### 3-FL

3-Fucosyllactose (3-FL) is a trisaccharide, more precisely, fucosylated, neutral trisaccharide composed of D-galactose, L-fucose,and D-glucose (Galβ1-4(Fucα1-3)Glc). It is naturally present in human milk. In a genetically modified cell or in an enzymatic reaction, 3-FL is produced primarily by an α1,3-fucosyltransferase or α1,3/4-fucosyltransferase enzymatic reaction with lactose and a fucosyl doner.

### LNT

Lacto-N-tetraose (LNT) is a tetrasaccharide, more precisely, a neutral tetrasaccharide composed of galactose, N-acetylglucosamine, galactose, and glucose (GlcNAcβ1-3Galβ1-4Glc). It is naturally present in human milk.

### DFL

Difucosyllactose (DFL or 2′,3-di-O-fucosyllactose) is an oligosaccharide, more precisely, focusylated neutral tetrasaccharide composed of L-fucose, D-galactose, L-fucose, and D-glucose (Fucα1-2Galβ1-4(Fucα1-3)Glc). It is naturally present in human milk. In a genetically modified cell or in an enzymatic reaction, DFL is produced primarily by an α1,2-fucosyltransferase, α1,3-fucosyltransferase and/or α1,3/4-fucosyltransferase enzymatic reaction with lactose and two fucosyl doners.

### 3′-SL and 6′-SL

3′-Sialyllactose and 6′-Sialyllactose are oligosaccharides, more precisely, sialylated trisaccharides composed of N-acetylneuraminyl, galactose, and glucose (Neu5Ac-α2-3Galβ1-4-Glc or Neu5Ac-α2-6Galβ1-4-Glc). They are naturally occurring in human milk. The specific functional benefits of 3′-SL include reducing the risk of infection by inhibiting the adhesion of pathogenic bacteria e.g., Helicobacter pylori and their toxins or viruses e.g., Rotavirus. 3′-SL and 6′-SL in particular promote brain development in infants by supplying sialic acid, an essential building block for neurons.

### FSL (3′-S, 3-FL)

3′-sialyl-3-fucosyllactose is an oligosaccharide, more precisely a sialylated and fucosylated tetrasaccharide composed of N-acetylneuraminic acid, D-galactose, L-fucose, and D-glucose units (Neu5Ac-α2-3Galβ1-4(Fucα1-3)Glc). It is naturally present in human milk.

### LST-a

Sialyllacto-N-tetraose A is an oligosaccharide, more precisely a sialylated pentasaccharide composed of N-acetylneuraminic acid, D-galactose, N-acetylglucosamine, D-galactose, and D-glucose units (Neu5Ac-α2-3Galβ1-4-3GlcNAcβ1-3Galβ1-4Glc). It is naturally present in human milk.

### LST-b

Sialyllacto-N-tetraose B is an oligosaccharide, more precisely a sialylated pentasaccharide composed of D-galactose, N-acetylneuraminic acid, N-acetylglucosamine, D-galactose, and D-glucose units (Galβ1-3(Neu5Ac-α2-6)GlcNAcβ1-3Galβ1-4Glc). It is naturally present in human milk.

### LST-c

Sialyllacto-N-neotetraose C is an oligosaccharide, more precisely a sialylated pentasaccharide composed of N-acetylneuraminic acid, D-galactose, N-acetylglucosamine, D-galactose, and D-glucose (Neu5Ac-α2-6Galβ1-4GlcNAcβ1-3Galβ1-4Glc). It is naturally present in human milk.

### DS-LNT

Disialyllacto-N-tetraose is an oligosaccharide, more precisely a sialylated hexasaccharide composed of N-acetylneuraminic acid, D-galactose, N-acetylneuraminic acid, N-acetylglucosamine, D-galactose, and D-glucose units (Neu5Ac-α2-3Galβ1-4-3(Neu5Ac-α2-6)GlcNAcβ1-3Galβ1-4Glc). It is naturally present in human milk.

### S-pLNnH

Sialyl-para-lacto-N-neohexaose is an oligosaccharide, more precisely a sialylated heptasaccharide composed of N-acetynleuraminic acid, D-galactose, N-acetylglucosamine, D-galactose, N-acetylglucosamine, D-galactose, and D-glucose units (Neu5Ac-α2-3Galβ1-4GlcNAcβ1-3Galβ1-4GlcNAcβ1-3Galβ1-4Glc.

### S-LNnH-I

Sialyl-lacto-N-neohexaose I is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-3Galβ1-4GlcNAcβ1-6(Galβ1-4GlcNAcβ1-3Galβ1-4Glc).

### DS-F-LNH II

Disialyl-fucosyl-lacto-N-hexaose II is an oligosaccharide, more precisely a sialylated, fucosylated nonasaccharide composed of N-acetylneuraminic acid, D-galactose, N-acetylglucosamine, N-acetylneuraminic acid, D-galactose, N-acetylglucosamine, L-fucose, D-galactose, and D-glucose units (Neu5Ac-α2-3Galβ1-3(Neu5Ac-α2-6)GlcNAcβ1-3(Galβ1-4(Fucα1-3)GlcNAcβ1-6)Galβ1-4Glc).

### FS-LNnH-I

Fucosyl-sialyl-lacto-N-neohexaose I is an oligosaccharide, more precisely a sialylated, fucosylated octasaccharide composed of N-acetylneuraminic acid, D-galactose, N-acetylglucosamine, D-galactose, L-fucose, N-acetylglucosamine, D-galactose, and D-glucose units (Neu5Ac-α2-6Galβ1-4GlcNAcβ1-3(Galβ1-4(Fucα1-3)GlcNAcβ1-6)Galβ1-4Glc).

### FS-LNH

Fucosyl-sialyl-lacto-N-hexaose is an oligosaccharide, more precisely a sialylated, fucosylated octasaccharide composed of N-acetylneuraminic acid, D-galactose, N-acetylglucosamine, L-fucose, D-galactose, N-acetylglucosamine, D-galactose, and D-glucose units (Neu5Ac-α2-6Galβ1-4GlcNAcβ1-6(Fucα1-2Galβ1-3GlcNAcβ1-3)Galβ1-4Glc).

### Fucosyl-LST-a (FLST-a)

Fucosyl-sialyllacto-N-tetraose A is an oligosaccharide, more precisely a sialylated, fucosylated hexasaccharide composed of N-acetylneuraminic acid, D-galactose, L-fucose, N-acetylglucosamine, D-galactose, and D-glucose units (Neu5Ac-α2-3Galβ1-3(Fucα1-4)GlcNAcβ1-3Galβ1-4Glc).

### Fucosyl-LST-b (FLST-b)

Fucosyl-sialyllacto-N-tetraose B is an oligosaccharide, more precisely a sialylated, fucosylated hexasaccharide composed of L-fucose, D-galactose, N-acetylneuraminic acid, N-acetylglucosamine, D-galactose, and D-glucose units (Fucα1-2Galβ1-3(Neu5Ac-α2-6)GlcNAcβ1-3Galβ1-4Glc).

### Fucosyl-LST-c (FLST-c)

Fucosyl-sialyllacto-N-neotetraose C is an oligosaccharide, more precisely a sialylated, fucosylated hexasaccharide composed of N-acetylneuraminic acid, D-galactose, N-acetylglucosamine, D-galactose, L-fucose, and D-glucose units (Neu5Ac-α2-6Galβ1-4GlcNAcβ1-3Galβ1-4(Fucα1-3)Glc.

### SLNH

Sialyl-lacto-N-hexaose is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-6Galβ1-4GlcNAcβ1-6(Galβ1-3GlcNAcβ1-3)Galβ1-4Glc).

### SLNnH-II

Sialyl-lacto-N-neohexaose II is an oligosaccharide, more precisely a sialylated heptasaccharide composed of N-acetylneuraminic acid, D-galactose, N-acetylglucosamine, D-galactose, N-acetylglucosamine, D-galactose, and D-glucose units (Neu5Ac-α2-6Galβ1-4GlcNAcβ1-3(Galβ1-4GlcNAcβ1-6)Galβ1-4Glc).

### Functional Enzymes for HMO Synthesis

To be able to synthesize one or more HMOs, the recombinant cell of the invention comprises at least one recombinant nucleic acid which encodes a functional enzyme with glycosyltransferase activity. The galactosyltransferase gene may be integrated into the genome (by chromosomal integration) of the genetically modified cell, or alternatively, it may be comprised in a plasmid DNA and expressed as plasmid-borne. If two or more glycosyltransferases are needed for the production of an HMO, e.g., LNT or LNnT, two or more recombinant nucleic acids encoding different enzymes with glycosyltransferase activity may be integrated in the genome and/or expressed from a plasmid, e.g., a β-1,3-N-acetylglucosaminyltransferase (a first recombinant nucleic acid encoding a first glycosyltransferase) in combination with a β-1,3-galactosyltransferase (a second recombinant nucleic acid encoding a second glycosyltransferase) for the production of LNT, where the first and second recombinant nucleic acid can independently from each other be integrated chromosomally or on a plasmid.

In one preferred embodiment, both the first and second recombinant nucleic acids are stably integrated into the chromosome of the production cell; in another embodiment at least one of the first and second glycosyltransferase is plasmid-borne. A protein/enzyme with glycosyltransferase activity (glycosyltransferase) may be selected in different embodiments from enzymes having the activity of α-1,2-fucosyltransferase, α-1,3-fucosyltransferase, α-1,3/4-fucosyltransferase, α-1,4-fucosyltransferase α-2,3-sialyltransferase, α-2,6-sialyltransferase, β-1,3-N-acetylglucosaminyltransferase, β-1,6-N-acetylglucosaminyltransferase, β-1,3-galactosyltransferase and β-1,4-galactosyltransferase. For example, the production of 2′-FL requires that the modified cell expresses an active α-1,2-fucosyltransferase enzyme. For the production of 3-FL the modified cell needs expression of an active α-1,3-fucosyltransferase enzyme. For the production of LNT the modified cell needs to express at least two glycosyltransferases, a β-1,3-N-acetylglucosaminyltransferase and a β-1,3-galactosyltransferase. For the production of 6′-SL the modified cell has to express an active α-2,6-sialyltransferase enzyme and a pathway for generating a sialate sugar nucleotide, such as a pathway for CMP-sialic acid synthesis, in particular synthesis of CMP-neu5A. For the production of 3′-SL the modified cell has to express an active α-2,3-sialyltransferase enzyme and a pathway for generating a sialate sugar nucleotide, such as a pathway for CMP-sialic acid synthesis, in particular in particular synthesis of CMP-neu5A. Some non-limiting embodiments of proteins having glycosyltransferase activity, which can be encoded by the recombinant genes comprised by the production cell, can be selected from non-limiting examples of Table 1.

TABLE 1
Glycosyltransferases for the production of neutral HMOs

| | Protein | | |
|---|---|---|---|
| | Sequence ID | | |
| Gene | (GenBank) | Description | HMO example |
| IgtA_Nm | WP_002248149.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Nm_MC58 | AAF42258.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Hd | AAN05638.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Ng_PID2 | AAK70338.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Ng_NCCP11945 | ACF31229.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Past | AAK02595.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Nc | EEZ72046.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| IgtA_Nm_87255 | ELK60643.1 | β-1,3-N- | LNT, LNnT, LNFP-I, LNFP-II, LNFP- |
| | | acetylglucosaminyl- | III, LNFP-V, LNFP-VI, LNDFH-I, |
| | | transferase | LNDFH-II, pLNH, F-pLNH I, pLNnH |
| galT_Hp/ | NP_207619.1 | ββ-1,4- | LNnT, LNFP-III, LNFP-VI, pLNH I, |
| HP0826 | | galactosyltransferase | F-pLNH I, pLNnH |
| galT_Nm/ | AAF42257.1 | β-1,4- | LNnT, LNFP-III, LNFP-VI, pLNH I, |
| IgtB | | galactosyltransferase | F-pLNH I, pLNnH |
| wbgO | WP_000582563.1 | β-1,3- | LNT, LNFP-I, LNFP-II, LNFP-V, |
| | | galactosyltransferase | LNDFH-I, LNDFH-II, pLNH, F-pLNH I |
| cpslBJ | AB050723.1 | ββ-1,3- | LNT, LNFP-I, LNFP-II, LNFP-V, |
| | | galactosyltransferase | LNDFH-I, LNDFH-II, pLNH, F-pLNH I |
| jhp0563 | AEZ55696.1 | β-1,3- | LNT, LNFP-I, LNFP-II, LNFP-V, |
| | | galactosyltransferase | LNDFH-I, LNDFH-II, pLNH, F-pLNH I |
| galTK | homologous to | β-1,3- | LNT, LNFP-I, LNFP-II, LNFP-V, |
| | BD182026 | galactosyltransferase | LNDFH-I, LNDFH-II, pLNH, F-pLNH I |
| futC | WP_080473865.1 | α-1,2-fucosyl- | 2′-FL, DFL, LNFP-I, LNDFH-I |
| | | transferase | |
| FucT2_HpUA802 | AAC99764.1 | α-1,2-fucosyl- | 2′-FL, DFL, LNFP-I, LNDFH-I |
| | | transferase | |
| FucT2_EcO126t | ABE98421.1 | α-1,2-fucosyl- | 2′-FL, DFL, LNFP-I, LNDFH-I |
| | | transferase | |
| FucT2_Hm12198 | CBG40460.1 | α-1,2-fucosyl- | 2′-FL, DFL, LNFP-I, LNDFH-I |
| | | transferase | |
| FucT2_Pm9515 | ABM71599.1 | α-1,2-fucosyl- | 2′-FL, DFL, LNFP-I, LNDFH-I |
| | | transferase | |
| FucT2_HpF57 | BAJ59215.1 | α-1,2-fucosyl- | 2′-FL, DFL, LNFP-I, LNDFH-I |
| | | transferase | |
| FucT6_3_Bf | CAH09151.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| FucT7_3_Bf | CAH09495.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| FucT_3_Am | ACD04596.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| MAMA_R764 | AGC02224.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| Mg791 | AEQ33441.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| Moumou_00703 | YP_007354660 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| futA | NP_207177.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| fucT | AAB81031.1 | α-1,3-fucosyl- | 2′-FL, 3-FL, DFL, LNFP-I, LNFP-III, |
| | | transferase | LNFP-V, LNFP-VI, LNDFH-II, F- |
| | | | pLNH I |
| fucTIII | AY450598.1 | α-1,4-fucosyl- | LNDFH-I, LNDFH-II |
| | | transferase | |
| fucTa | AF194963.1 | α-1,3/4-fucosyl- | LNFP-II, LNDFH-I, LNDFH-II |
| | | transferase | |

TABLE 2
Glycosyltransferases for the production of sialylated HMOs

| | Protein | | |
|---|---|---|---|
| | Sequence ID | | |
| Gene | (GenBank) | Description | HMO example |
| Pd2, 6ST | BAA25316.1 | α-2,6-sialyltransferase | 6′-SL |
| PspST6 | BAF92026.1 | α-2,6-sialyltransferase | 6′-SL |
| PiST6_145 | BAF91416.1 | α-2,6-sialyltransferase | 6′-SL |
| PiST6_119 | BAI49484.1 | α-2,6-sialyltransferase | 6′-SL |
| NST | AAC44541.1 | α-2,3-sialyltransferase | 3′-SL |
| Cst-III | AAK85419.1 | α-2,3-sialyltransferase | 3′-SL |
| Cst-II | AAG43979.1 | α-2,3-sialyltransferase | 3′-SL |
| Cst-I | AAF13495.1 | α-2,3-sialyltransferase | 3′-SL |
| U60664.1 | AAC44539.1 | α-2,3-sialyltransferase | 3′-SL |
| CST-II | ABF67963.1 | α-2,3-sialyltransferase | 3′-SL |

In one embodiment, the genetically modified cell of the invention is modified to heterologously express a α-2,3-sialyl-transferase as shown in SEQ ID NO: 3 and/or a functional homologue thereof having an amino acid sequence which is at least 70% identical to SEQ ID NO: 3, such as at least 80%, such as at least 90%, such as at least 95% such as at least 99% or such as 100%. In addition, the genetically modified cell contains a biosynthetic pathway for making a sialate sugar nucleotide.

In another embodiment, the genetically modified cell of the invention is modified to heterologously express a α-2,6-sialyl-transferase as shown in SEQ ID NO: 4 and/or a functional homologue thereof having an amino acid sequence which is at least 70% identical to SEQ ID NO: 4, such as at least 80%, such as at least 90%, such as at least 95% such as at least 99% or such as 100%. In addition, the genetically modified cell contains a biosynthetic pathway for making a sialate sugar nucleotide.

The biosynthetic pathway for making a sialate sugar nucleotide may either be inherent in the cell or provided by one or more heterologous genes such as the neuBCA gene cluster of SEQ ID NO: 17, wherein the heterologous CMP-Neu5Ac synthetase is encoded by neuA of SEQ ID NO: 16, the heterologous sialic acid synthase is encoded by neuB of SEQ ID NO: 12, and the heterologous GlcNAc-6-phosphate 2 epimerase is encoded by neuC of SEQ ID NO: 14.

In one embodiment, the Fred expressing genetically modified cell may further be modified to heterologously express a α-2,6-sialyl-transferase as shown in SEQ ID NO: 4 and/or a functional homologue thereof having an amino acid sequence which is at least 70% identical to SEQ ID NO: 4, such as at least 80%, such as at least 90%, such as at least 95% such as at least 99% or such as 100%. In addition, the genetically modified cell contains a biosynthetic pathway for making a sialate sugar nucleotide. This may either be inherent in the cell or provided by one or more heterologous genes such as the neuBCA gene cluster of SEQ ID NO: 17, wherein the heterologous CMP-Neu5Ac synthetase is encoded by neuA of SEQ ID NO: 16, the heterologous sialic acid synthase is encoded by neuB of SEQ ID NO: 12, and the heterologous GlcNAc-6-phosphate 2 epimerase is encoded by neuC of SEQ ID NO: 14.

### Heterologous Nucleic Acid Sequence Encoding an MFS Transporter

An aspect of the present invention is the provision of a nucleic acid construct comprising a heterologous nucleic acid sequence(s) encoding a polypeptide capable of sugar transportation which is a major facilitator superfamily (MFS) polypeptide as shown in SEQ ID NO: 1, or a functional homologue thereof with an amino acid sequence which is more than 95.4% or 99.7% identical to SEQ ID NO: 1, wherein the nucleic acid sequence encoding the sugar transportation polypeptide has at least 70%, such as at least 80% sequence identity to SEQ ID NO: 2.

Preferably, the nucleic acid sequence of SEQ ID NO: 2, or of a homologue thereof, is regulated by a regulatory element, in particular an element selected from the group consisting of PglpF, PglpF_SD4 and PglpF_SD7. In a further embodiment, the nucleic acid construct further comprises as nucleic acid sequence encoding a sialyl-transferase, especially a sialyl-transferase selected from table 2 herein.

By the term “heterologous nucleic acid sequence”, “recombinant gene/nucleic acid/DNA encoding” or “coding nucleic acid sequence” is meant an artificial nucleic acid sequence (i.e. produced in vitro using standard laboratory methods for making nucleic acid sequences) that comprises a set of consecutive, non-overlapping triplets (codons) which is transcribed into mRNA and translated into a polypeptide when under the control of the appropriate control sequences, i.e. a promoter. The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleotide sequences encoding a given protein may be produced. The term nucleic acid is used interchangeably with the term “polynucleotide”.

An “oligonucleotide” is a short chain nucleic acid molecule.

“Primer” is an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification but may alternatively be double stranded. If double stranded, the primer is first treated to separate its strands before being used to prepare extension products. Preferably, the primer is a deoxyribonucleotide sequence. The primer must be sufficiently long to prime the synthesis of extension products in the presence of the inducing agent. The exact lengths of the primers will depend on many factors, including temperature, source of primer and the use of the method.

The recombinant nucleic sequence of the invention may be a coding DNA sequence, e.g., a gene, or non-coding DNA sequence, e.g., a regulatory DNA, such as a promoter sequence. One aspect of the invention relates to providing a recombinant cell comprising recombinant DNA sequences encoding enzymes necessary for the production of one or more HMOs and a DNA sequence encoding Fred transporter. Accordingly, in one embodiment the invention relates to a nucleic acid construct comprising a coding nucleic sequence, i.e. recombinant DNA sequence of a gene of interest, e.g., a glycosyltransferase gene or the fred gene, and a non-coding DNA sequence, e.g., a promoter DNA sequence, e.g., a recombinant promoter sequence derived from the promoter of lac operon or an glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. Typically, it refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting.

In one embodiment, the nucleic acid construct of the invention may be a part of the vector DNA, in another embodiment the construct it is an expression cassette/cartridge that is integrated in the genome of a host cell. Accordingly, the term “nucleic acid construct” means an artificially constructed segment of nucleic acid, in particular a DNA segment, which is intended to be ‘transplanted’ into a target cell, e.g., a bacterial cell, to modify expression of a gene of the genome or express a gene/coding DNA sequence which may be included in the construct. In the context of the invention, the nucleic acid construct contains a recombinant DNA sequence comprising two or more recombinant DNA sequences: essentially, a non-coding DNA sequence comprising a promoter DNA sequence and a coding DNA sequence encoding a gene of interest, e.g., Fred protein, a glycosyltransferase, of another gene useful for production of an HMO in a host cell. Preferably, the construct comprises further non-coding DNA sequences that either regulate transcription or translation of the coding DNA of the construct, e.g., a DNA sequence facilitating ribosome binding to the transcript, a leading DNA sequence that stabilize the transcript.

Integration of the recombinant nucleic acid of interest comprised in the construct (expression cassette) into the bacterial genome can be achieved by conventional methods, e.g., by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998);180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005);71(4):1829-35); or positive clones, i.e., clones that carry the expression cassette, can be selected e.g., by means of a marker gene, or loss or gain of gene function.

A single copy of the expression cassette comprising a gene of interest may be sufficient to secure production of a desired HMO and achieve the desired effects according to the invention. Accordingly, in some preferred embodiments, the invention relates to a recombinant HMO producing cell that comprises one, two or three copies of a gene of interest integrated in the genomic DNA of the cell. In some embodiments the single copy of the gene is preferred.

In one preferred embodiment, recombinant coding nucleic acid sequence of the nucleic acid construct of the invention is heterologous with respect to the promoter, which means that in the equivale native coding sequence in the genome of species of origin is transcribed under control of another promoter sequence (i.e., not the promoter sequence of the construct). Still, with respect to the host cell, the coding DNA may be either heterologous (i.e., derived from another biological species or genus), such as e.g., the DNA sequence encoding Fred protein expressed in Escherichia coli host cells, or homologous (i.e., derived from the host cell), such as e.g., genes of the colonic acid operon, the wca genes.

Preferably, the construct of the invention comprising a gene related to biosynthetic production of an HMO, a promoter DNA sequence, and other regulatory sequences, such as a ribosomal binding site sequence (e.g., Shine-Dalgarno sequence), expressed in the genetically modified cell enables production of the HMO at the level of at least 0.03 g/OD (optical density) of 1 litre of the fermentation media comprising a suspension of genetically modified cell s, e.g., at the level of around 0.05 g/l/OD to around 0.5 g/l/OD. For the purposes of the invention, the later level of HMO production is regarded as “sufficient” and the genetically modified cell capable of producing this level of a desired HMO is regarded as “suitable genetically modified cell”, i.e., the cell can be further modified to express the HMO transporter protein, e.g., Fred, to achieve at least one effect described herein that is advantageous for the HMO production.

The genetically modified cell or the nucleic acid construct of the present invention comprises a nucleic acid sequence such as a heterologous gene encoding a putative Fred MFS (major facilitator superfamily) transporter protein.

A nucleic acid construct of the present invention therefore contains a nucleic acid sequence having at least 70% sequence identity to the gene, fred, SEQ ID NO: 2, and which is capable of encoding a functional MFS transporter. Preferably the nucleic acid sequence of SEQ ID NO: 2, or a homologue thereof, is regulated by a regulatory element, in particular an element selected from the group consisting of PglpF, PglpF_SD4 and PglpF_SD7. In a further embodiment the nucleic acid construct further comprises as nucleic acid sequence encoding a sialyl-transferase, especially a sialyl-transferase selected from table 2 herein.

The nucleic acid sequence contained in the genetically modified cell or in the nucleic acid construct encodes for a protein of SEQ ID NO: 1, or a functional homologue thereof with an amino acid sequence which is more than 70% identical to SEQ ID NO: 1.

The nucleic acid sequence contained in the genetically modified cell or in nucleic acid construct encodes for a protein of SEQ ID NO: 1, or a functional homologue thereof with an amino acid sequence which is more than 95.4% identical to SEQ ID NO: 1.

A functional homologue of the protein of SEQ ID NO: 1, may be obtained by mutagenesis. The functional homologue should have a remaining functionality of at least 50%, such as 60%, 70%, 80%, 90% or 100% compared to the functionality of amino acid sequence of SEQ ID NO: 1. The functional homologue can have a higher functionality compared to the functionality of amino acid sequence of SEQ ID NO: 1. The functional homologue of SEQ ID NO: 1, should be able to enhance HMO production of the genetically modified cell according to the invention.

### A Genetically Modified Cell

A “genetically modified cell” as used herein is understood as a cell whose genetic material has been altered by human intervention using a genetic engineering technique, such a technique is for example, but not limited to, transformation or transfection e.g., with a heterologous polynucleotide sequence, Crisper/Cas editing and/or random mutagenesis. In the present context, the terms a “genetically modified cell” and “a host cell” are used interchangeably.

In the present invention the “genetically modified cell” is preferably a host cell which has been transformed or transfected by an exogenous polynucleotide sequence.

The genetically modified cell is preferably a prokaryotic cell. Appropriate microbial cells that may function as a host cell include yeast cells, bacterial cells, archaebacterial cells, algae cells, and fungal cells.

The genetically engineered cell may be e.g., a bacterial or a fungus. In one preferred embodiment, the genetically engineered cell is a bacterial cell.

### Host Cell

The genetically modified cell (host cell or recombinant cell) may be e.g., a bacterial or yeast cell. In one preferred embodiment, the genetically modified cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be modified using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichfi are also suitable bacterial species for the invention described herein. Also included as part of this invention are strains, modified as described here, from the genera Enterococcus (e.g., Enterococcus faecium and Enterococcus thermophiles), Bifidobacterium(e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Bacteria comprising the characteristics described herein are cultured in the presence of lactose, and an oligosaccharide, such as an HMO, produced by the cell is retrieved, either from the bacterium itself or from a culture supernatant of the bacterium. In one preferred embodiment, the genetically modified cell of the invention is an Escherichia coli cell.

In another preferred embodiment the host cell is a yeast cell e.g., Saccharomyces cerevisiae, Schizosaccharomyces pombe, Pichia pastoris, Kluveromyces lactis, Kluveromyces marxianus, etc.

In another embodiment the host cell is a filamentous fungi such as Aspargillus sp, Fusarium sp or Thricoderma sp, exemplary species are A. niger, A. nidulans, A. oryzae, F. solani, F. graminearum and T. reesei.

Genetically modified cells of the invention can be provided using standard methods of the art e.g., those described in the manuals by Sambrook et al., Wilson & Walker, “Maniatise et al., and Ausubel et al.

A host suitable for the HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI:41901)). For the purposes of the invention, an HMO-producing host cell is genetically manipulated to either comprise any p-galactosidase gene or to comprise the gene that is inactivated. The gene may be inactivated by a complete or partial deletion of the corresponding nucleic acid sequence from the bacterial genome, or the gene sequence is mutated in the way that it is transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

In some embodiments, the engineered cell, e.g., bacterium, contains a deficient sialic acid catabolic pathway. By “sialic acid catabolic pathway” is meant a sequence of reactions, usually controlled and catalyzed by enzymes, which results in the degradation of sialic acid. An exemplary sialic acid catabolic pathway described herein is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1(GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745, incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanK are mutated, while nanE remains functional. In another preferred embodiment, nanA and nanE are mutated, while nanK has not been mutated, inactivated or deleted. A mutation is one or more changes in the nucleic acid sequence coding the gene product of nanA, nanK, nanE, and/or nanT. For example, the mutation may be 1, 2, up to 5, up to 10, up to 25, up to 50 or up to 100 changes in the nucleic acid sequence. For example, the nanA, nanK, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. A “not mutated” gene or protein does not differ from a native, naturally-occurring, or endogenous coding sequence by 1, 2, up to 5, up to 10, up to 20, up to 50, up to 100, up to 200 or up to 500 or more codons, or to the corresponding encoded amino acid sequence.

In a preferred embodiment the bacterium (e.g., E. coli) comprises a sialic acid synthetic capability, i.e. the genetically modified cell comprises a biosynthetic pathway for making a sialate sugar nucleotide. For example, the genetically modified bacterium comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

In one embodiment the genetically modified cell comprises an UDP-GlcNAc 2-epimerase of SEQ ID NO: 13 (NeuC) and/or a CMP-Neu5Ac synthetase of SEQ ID NO: 11 (NeuB) and/or a Neu5Ac synthase of SEQ ID NO: 15 (NeuA), or a functional variant thereof having an amino acid sequence which is at least 80% identical, such as at least 90% or such as at least 99% to SEQ ID NOs: 11 or 13 or 15, respectively.

Production of neutral N-acetylglucosamine-containing HMOs in modified bacteria is also known in the art (see e.g., Gebus C et al. (2012) Carbohydrate Research 363 83-90).

For the production of N-Acetylneuraminic acid (sialyl) containing HMOs, such as 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT), sialyl-para-lacto-N-neohexaose (S-pLNnH), sialyl-lacto-N-neohexaose I (S-LNnH-I), disialyl-fucosyl-lacto-N-hexaose II (DS-F-LNH II), fucosyl-sialyl-lacto-N-neohexaose I (FS-LNnH-I) and/or fucosyl-sialyl-lacto-N-hexaose (FS-LNH) said genetically modified cell is modified to comprise an exogeneous N-Acetylneuraminic acid transferase (i.e. a sialyl transferase), or a functional variant or fragment thereof. An exogenous sialyl transferase gene may be obtained from any one of a number of sources, such as but not limited to sialyl transferases listed in Table 2.

For the production of N-acetylglucosamine-containing HMOs, such as Lacto-N-triose 2 (LNT-2), Lacto-N-tetraose (LNT), Lacto-N-neotetraose (LNnT), Lacto-N-fucopentaose I (LNFP-I), Lacto-N-fucopentaose II (LNFP-II), Lacto-N-fucopentaose III (LNFP-III), Lacto-N-fucopentaose V (LNFP-V), Lacto-N-difucohexaose I (LDFH-I), Lacto-N-difucohexaose II (LDFH-II), and Lacto-N-neodifucohexaose II (LNDFH-III), as described above, and it is modified to comprise an exogenous UDP-GlcNAc:Galα/β-R β-3-N-acetylglucosaminyltransferase gene, or a functional variant or fragment thereof. This exogenous UDP-GlcNAc:Galα/β-R β-3-N-acetylglucosaminyltransferase gene may be obtained from any one of a number of sources, e.g., the IgtA gene described from N. meningitides (Genbank protein Accession AAF42258.1) or N. gonorrhoeae (Genbank protein Accession ACF31229.1). Optionally, an additional exogenous glycosyltransferase gene may be co-expressed in the bacterium comprising an exogenous UDP-GlcNAc:Galα/β-R β-3-N-acetylglucosaminyltransferase. For example, a β-1,4-galactosyltransferase gene is co-expressed with the UDP-GlcNAc:Galα/β-R β-3-N-acetylglucosaminyltransferase gene. This exogenous β-1,4-galactosyltransferase gene can be obtained from any one of a number of sources, e.g., the one described from N. meningitidis, the IgtB gene (Genbank protein Accession AAF42257.1), or from H. pylori, the HP0826/ga/T gene (Genbank protein Accession NP_207619.1). Optionally, the additional exogenous glycosyltransferase gene co-expressed in the bacterium comprising an exogenous UDP-GlcNAc:Galα/β-R β-3-N-acetylglucosaminyltransferase gene is a P-I,3-galactosyltransferase gene, e.g., that described from E. coli 055:H7, the wbgO gene (Genbank protein Accession WP_000582563.1), or from H. pylori, the jhp0563 gene (Genbank protein Accession AEZ55696.1), or from Streptococcus agalactiae type Ib O12 the cpslBJ gene (Genbank protein Accession AB050723). Functional variants and fragments of any of the enzymes described above are also encompassed by the disclosed invention.

A sialyl transferase gene, N-acetylglucosaminyltransferase gene and/or a galactosyltransferase gene, can also be operably linked to a Pglp promotor and be expressed from the corresponding genome-integrated cassette. In one embodiment, the gene that is gnomically integrated is a gene encoding for a galactosyltransferase, e.g., HP0826 gene encoding for the GaIT enzyme from H. pylori (Genbank protein Accession NP_207619.1); in another embodiment, the gene that is genomically integrated is a gene encoding a β-1,3-N-acetylglucosaminyltransferase, e.g., IgtA gene from N. meningitidis (Genbank protein Accession AAF42258.1); In a presently preferred embodiment, the gene that is gnomically integrated is a gene encoding a α-2,3-sialyltransferase e.g, NST of Neisseria meningitidis (Genbank protein asession AAC44541.1 or SEQ ID NO: 3). In these embodiments the gene encoding a β-1,3-N-acetylglucosaminyltransferase or galactosyltransferase, correspondingly, may either be expressed from a genome-integrated or plasmid borne cassette. The second gene may optionally be expressed either under the control of a glp promotor (SEQ ID NO: 5) or under the control of any other promotor suitable for the expression system, e.g., Plac (SEQ ID NO: 8).

HMO producing host cells typically comprise a functional lacY and a dysfunctional lacZ gene.

The HMOs produced by recombinant cells of the invention may be purified using a suitable procedure available in the art (e.g., such as described in WO2015/188834, WO2017/182965 or WO2017/152918).

### Encoded Polypeptide Capable of Sugar Transportation

Sugar transportation relates to the transport of a sugar, such as, but not limited to, an oligosaccharide, and in relation to the invention, influx and/or efflux transport of one/or more HMOs, from the cytoplasm or periplasm of a genetically modified cell to the production media and/or from the production media to the cytoplasm or periplasm of a genetically modified cell. Thus, a polypeptide expressed in the genetically modified cell, capable of transporting HMOs from the cytoplasm or periplasm to the production medium and/or from the production media to the cytoplasm or periplasm of a genetically modified cell is a polypeptide capable of sugar transportation. Thus, in the present invention, sugar transportation can mean efflux and/or influx transportation of sugar, such as, but not limited to, an oligosaccharide.

In that regard, the polypeptide capable of sugar transportation is a polypeptide belonging to the Major Facilitator Superfamily (MFS). Essentially, the polypeptide has more than 70%, such as at least 80%, such as at least 90%, 95%, 99% or 99.9% sequence identity with SEQ ID NO:1 or it is a functional variant thereof as described herein. In particular, the polypeptide has more than 95.4%, such as at least 95.5%, such as at least 96%, 97%, 98% or 99% sequence identity with SEQ ID NO:1 or it is a functional variant thereof as described herein. SEQ ID NO: 1 is the amino acid sequence of the Fred protein.

The genetically modified cell or the nucleic acid construct of the present invention comprises a nucleic acid sequence such as a heterologous gene encoding a Fred protein. Said nucleic acid sequence has at least 70% sequence identity to the fred gene as shown in SEQ ID NO: 2, such as at least 75%, 80%, 85%, 90%, 95% or 99%.

In one embodiment, the nucleic acid sequence construct encodes a protein of SEQ ID NO: 1, or a functional homologue thereof with an amino acid sequence which is more than 70%, such as at least 80%, 90%, 95%, 99%, or 99.9% identical to SEQ ID NO: 1.

In another embodiment, the nucleic acid sequence construct encodes a protein of SEQ ID NO: 1, or a functional homologue thereof with an amino acid sequence which is more than 95.4%, such as at least 95.5%, 96%, 97%, 98%, or 99% identical to SEQ ID NO: 1.

A functional homologue of the protein of SEQ ID NO: 1, may be obtained by mutagenesis. The functional homologue should have a remaining functionality of at least 50%, such as 60%, 70%, 80%, 90% or 100% compared to the functionality of amino acid sequence of SEQ ID NO: 1. The functional homologue can have a higher functionality compared to the functionality of amino acid sequence of SEQ ID NO: 1. The functional homologue of SEQ ID NO: 1, should be able to enhance HMO production of the genetically modified cell according to the invention. In particular the functional homologue of SEQ ID NO: 1, should be able to enhance the ratio of sialylated HMO outside the cells of the genetically modified cell according to the invention.

The genetically modified cell or the nucleic acid construct may contain one or more nucleic acid sequences encoding the polypeptide capable of sugar transportation. More often, the genetically modified cell or the nucleic acid construct of the invention, encodes a single copy of a polypeptide capable of sugar transportation.

A single copy of the expression cassette comprising a gene of interest may be sufficient to secure production of a desired HMO and achieve the desired effects according to the invention. Accordingly, in some preferred embodiments, the invention relates to a recombinant HMO producing cell that comprises one, two or three copies of a gene or genes of interest integrated in the genomic DNA of the cell. In some embodiments the single copy of the gene or genes is/are preferred.

The genetically modified cell or the nucleic acid construct, may also contain one or more regulatory elements for the regulation of the expression of a nucleic acid sequence encoding the sugar transportation polypeptide and wherein said nucleic acid sequence has at least 70% sequence identity with SEQ ID NO: 2.

### Nucleic Acid Constructs

For the transfection or transformation of the host cell nucleic acid constructs will be needed. These can either exist as plasmid constructs within the cell or be suited for genomic integration.

One aspect of the present invention is a nucleic acid construct comprising: i) a nucleic acid sequence encoding an MFS polypeptide according to SEQ ID NO: 1, or a functional homologue thereof, having at least 70% sequence identity to SEQ ID NO: 1, such as 80%, such as 90%, such as 95%, such as 99.7% such as 100% sequence identity to SEQ ID NO: 1, wherein the nucleic acid sequence encoding the MFS polypeptide has at least 70% sequence identity to SEQ ID NO: 2 such as 80%, such as 90%, such as 95% such as 100% sequence identity to SEQ ID NO: 2, and/or ii) a nucleic acid sequence encoding one or more polypeptide(s) with sialyl-transferase capabilities, such as a polypeptide of SEQ ID NO: 3 or SEQ ID NO: 4 or a functional variant thereof which has least 70%, such as 80%, such as 90%, such as 95% to SEQ ID NO: 3 or SEQ ID NO: 4 and iii) a nucleic acid sequence comprising a regulatory element that regulates the expression the nucleic acid sequence(s) of point i) and/or ii).

In one embodiment of the invention, the nucleic acid construct comprises: i) a nucleic acid sequence encoding an MFS polypeptide according to SEQ ID NO: 1, or a functional homologue thereof, having at least 70% sequence identity to SEQ ID NO: 1, such as 80%, such as 90%, such as 95% such as 100% sequence identity to SEQ ID NO: 1, wherein the nucleic acid sequence encoding the MFS polypeptide has at least 70% sequence identity to SEQ ID NO: 2 such as 80%, such as 90%, such as 95% such as 100% sequence identity to SEQ ID NO: 2, and ii) a nucleic acid sequence encoding one or more polypeptide(s) with sialyl-transferase capabilities, such as a polypeptide of SEQ ID NO: 3 or SEQ ID NO: 4 or a functional variant thereof or a functional variant thereof which has least 70%, such as 80%, such as 90%, such as 95% to SEQ ID NO: 3 or SEQ ID NO: 4 and iii) a nucleic acid sequence comprising a regulatory element that regulates the expression of both the nucleic acid sequence(s) of point i) and ii).

In one embodiment of the invention the nucleic acid construct comprises: i) a nucleic acid sequence encoding an MFS polypeptide according to SEQ ID NO: 1, or a functional homologue thereof, having at least 70% sequence identity to SEQ ID NO: 1, such as 80%, such as 90%, such as 95% such as 100% sequence identity to SEQ ID NO: 1, wherein the nucleic acid sequence encoding the MFS polypeptide has at least 70% sequence identity to SEQ ID NO: 2 such as 80%, such as 90%, such as 95% such as 100% sequence identity to SEQ ID NO: 2, and ii) a nucleic acid sequence comprising a regulatory element that regulates the expression of both the nucleic acid sequence(s) of point i).

In one embodiment of the invention the nucleic acid construct comprises: i) a nucleic acid sequence encoding one or more polypeptide(s) with sialyl-transferase capabilities, such as a polypeptide of SEQ ID NO: 3 or SEQ ID NO: 4 or a functional variant thereof or a functional variant thereof which has least 70%, such as 80%, such as 90%, such as 95% to SEQ ID NO: 3 or SEQ ID NO: 4 and ii) a nucleic acid sequence comprising a regulatory element that regulates the expression of both the nucleic acid sequence(s) of point i) and ii).

In one embodiment of the invention the nucleic acid construct comprises: i) a nucleic acid sequence encoding an MFS polypeptide according to SEQ ID NO: 1, or a functional homologue thereof, having at least 70% sequence identity to SEQ ID NO: 1, such as 80%, such as 90%, such as 95% such as 100% sequence identity to SEQ ID NO: 1, wherein the nucleic acid sequence encoding the MFS polypeptide has at least 70% sequence identity to SEQ ID NO: 2 such as 80%, such as 90%, such as 95% such as 100% sequence identity to SEQ ID NO: 2, and ii) a nucleic acid sequence encoding one or more polypeptide(s) with sialyl-transferase capabilities, such as a polypeptide of SEQ ID NO: 3 or SEQ ID NO: 4 or a functional variant thereof or a functional variant thereof which has least 70%, such as 80%, such as 90%, such as 95% to SEQ ID NO: 3 or SEQ ID NO: 4 and iii) at least two nucleic acid sequences comprising a regulatory element that independently regulate the expression of the nucleic acid sequences of point i) and ii).

The regulatory element of the nucleic acid construct of the present invention is preferably selected from the group consisting of PglpF, PglpF_SD4 and PglpF_SD7.

### Regulatory Element

As mentioned above, the genetically modified cell or the nucleic acid construct may further comprise a nucleic acid sequence comprising a regulatory element for the regulation of the expression of a nucleic acid sequences, such as, but not limited to, the nucleic acid sequence having at least 70% sequence identity to SEQ ID NO: 2, a sialyltransferase of table 2 and/or alternative transferases of table 1. The nucleic acid sequence of the regulatory region may be heterologous or homologous.

The term, a “regulatory element” or “promoter” or “promoter region” or “promoter element” is a nucleic acid sequence that is recognized and bound by a DNA dependent RNA polymerase during initiation of transcription. The promoter, together with other transcriptional and translational regulatory nucleic acid sequences (also termed “control sequences”) is necessary to express a given gene or group of genes (an operon). In general, the transcriptional and translational regulatory sequences include, but are not limited to, promoter sequences, ribosomal binding sites, transcriptional start and stop sequences, translational start and stop sequences, and enhancer or activator sequences. The “transcription start site” means the first nucleotide to be transcribed and is designated +1. Nucleotides downstream of the start site are numbered +2, +3, +4 etc., and nucleotides in the 5′ opposite (upstream) direction are numbered −1, −2, −3 etc. The promoter of the construct can derive from a promoter region of any gene encoded in the genome of a species. Preferably, a promoter region of the genomic DNA of E. coli. Accordingly, any promoter that is able to bind to an RNA polymerase and initiate transcription is suitable for practicing the invention. In principle, any promoter can be used to control transcription of the recombinant gene, such as the MFS transporter or the glycosyltransferases of the invention. In carrying out the invention, different or identical promoter sequences may be used to drive transcription of different genes of interest integrated in the genome of the host cell or in expression vector DNA. In one example promoter sequence A is promoting the expression of the MFS transporter, and another promoter sequence B or identical promoter sequence A is promoting the expression of the glycosyltransferase.

To have an optimal expression of the recombinant genes included in the construct, the construct may comprise further regulatory sequences, e.g., a leading DNA sequence, such as a DNA sequence derived from 5′-untranslated region (5′UTR) of a glp gene of E. coli, a sequence for ribosomal binding. Examples of the later sequences are described in WO2019/123324 (incorporated herein by reference) and are illustrated in non-limiting working examples herein.

In one aspect of the invention, one or more regulatory elements may be inserted into to the DNA construct encoding the fred gene according to SEQ ID NO: 2 and/or into to the DNA construct encoding one or more glycosyltransferases, according to the invention. In another aspect of the invention, one or more copies of the fred gene according to SEQ ID NO: 2 may be inserted into the DNA construct, according to the invention. In yet another aspect of the invention, the fred gene according to SEQ ID NO: 2 may be inserted into one or more identical or non-identical DNA constructs, according to the invention.

In one aspect of the invention, the regulatory element for the regulation of the expression of a recombinant gene included in the construct of the invention is glpFKX operon promoter, PglpF (SEQ ID NO: 5). In another aspect of the invention, the promoter is lac operon promoter, Plac (SEQ ID NO: 8). And in yet another aspect of the invention, the regulatory element is PglpF_SD4 (SEQ ID NO: 6) and/or PglpF_SD7 (SEQ ID NO: 7), which are modified version of the PglpF sequence comprising a modified ribosomal binding site sequence downstream of the promoter sequence. However, any promoter enabling transcription and/or regulation of the level of transcription, of one or more recombinant nucleic acids that encode one or more polypeptides according to the invention are suitable for practicing the invention.

Typically, promoters for the expression of a heterologous gene according to the present invention are selected from table 3.

TABLE 3

| | | | SEQ ID NO |
|---|---|---|---|
| | | | in the |
| Promoter | | | present |
| name | Description | Reference | application: |
| PgatY_70UTR | E. coli promoter for gatYZABCD; tagatose-1,6- | NA | |
| | bisP aldolase | | |
| PglpF | E. coli promoter for glpFKX operon; glycerol | WO2019123324 | 5 |
| | uptake | | |
| PglpF_SD1 | E. coli promoter for glpFKX operon; glycerol | WO2019123324 | |
| | uptake | | |
| PglpF_SD10 | E. coli promoter for glpFKX operon; glycerol | WO2019123324 | |
| | uptake | | |
| PglpF_SD2 | E. coli promoter for glpFKX operon; glycerol | WO2019123324 | |
| | uptake | | |
| PglpF_SD3 | E. coli promoter for glpFKX operon; glycerol | WO2019123324 | |
| | uptake | | |
| PglpF_SD4 | E. coli promoter for glpFKX operon; glycerol | WO2019123324 | 6 |
| | uptake | | |
| PglpF_SD5 | E. coli promoter for glpFKX operon; glycerol | WO2019123324 | |
| | uptake | | |
| PglpF_SD6 | E. coli promoter for glpFKX operon; glycerol | WO2019123324 | |
| | uptake | | |
| PglpF_SD7 | E. coli promoter for glpFKX operon; glycerol | WO2019123324 | 7 |
| | uptake | | |
| PglpF_SD8 | E. coli promoter for glpFKX operon; glycerol | WO2019123324 | |
| | uptake | | |
| PglpF_SD9 | E. coli promoter for glpFKX operon; glycerol | WO2019123324 | |
| | uptake | | |
| Plac_16UTR | E. coli promoter for lacZYA: lac operon | WO2019123324 | |
| Plac | E. coli promoter for lacZYA: lac operon | WO2019123324 | 8 |
| PmglB_70UTR | E. coli promoter for mglBAC; galactose/methyl- | NA | |
| | galactosidade transporter | | |
| PmglB_70UTR_SD4 | E. coli promoter for mglBAC; galactose/methyl- | NA | |
| | galactosidade transporter | | |

The preferred regulatory elements present in a genetically modified cell or in a nucleic acid construct of the present invention, is selected from the group consisting of, PgatY_7OUTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, Plac_16UTR, Plac, PmglB_70UTR and PmglB_70UTR_SD4.

Especially preferred regulatory elements present in a genetically modified cell or in a nucleic acid construct of the present invention, is selected from the group consisting of PglpF, PglpF_SD4 and PglpF_SD7.

### Genetically Modified Cells for Biosynthetic Production

In the present invention, promoters may be either necessary or beneficial for achieving an optimal level of biosynthetic production of one or more HMOs in the genetically modified cell and allowing to achieve the desired effects according to the invention. Thus, a promoter sequence, of this invention, enables transcription and/or regulates the expression of a polypeptide capable of sugar transportation and/or the glycosyltransferases of the invention, resulting in optimized biosynthesis and transport of HMOs or HMO precursors and/or degradation of by-products of the HMO production.

In the genetically modified cell of the invention, a nucleic acid construct is comprised in the genetically modified cell, that encodes at least one gene related to biosynthetic production of one or more HMOs, a promoter DNA sequence, and other regulatory sequences, such as a ribosomal binding site sequence (e.g., Shine-Dalgarno sequence). The expression of the gene or genes related to the biosynthetic production of one or more HMOs in a genetically modified cell, enables production of the HMOs, making the host cell a “suitable host cell” for carrying out the invention as described. In the genetically modified cell, the expression, of the gene or genes related to the biosynthetic production of HMOs, as mentioned above, enables the production of one or more HMOs at level of 0.03 g/l/OD (optical density) from 1 liter of fermentation media comprising a suspension of the genetically modified cell s. Thus, the HMO level could be approx. 0.05 g/l/OD to approx. 0.5 g/l/OD, such as at least 0.4 g/L/OD. For the purposes of the invention, the level of HMO production is regarded as “sufficient” and the genetically modified cell capable of producing this level of a desired HMO or mixture of HMOs is regarded as the suitable genetically modified cell for carrying out the invention.

Thus, in the light of the invention, the “suitable genetically modified cell ” can be further modified, as described above, to express the sugar polypeptide capable of sugar transportation of the MFS family, e.g., fred, to achieve an, in one way or another, advantageous HMO production, such as but not limited to, a higher HMO level or biosynthetic production, a higher level of purity in the biosynthetic production, a faster production time and/or a more efficient biosynthetic production of HMOs.

### Method of Production

An aspect of the invention is related to a method for the production of one or more sialylated HMOs, the method comprising the steps of:
a) providing a genetically modified cell capable of producing an HMO, wherein said cell comprises
   i. a recombinant nucleic acid encoding a protein of SEQ ID NO: 1, or a functional homologue thereof with an amino acid sequence which is more than 70%, such as at least 80% identical, preferably at least 90% identical, more preferably at least 94.5%, most preferably at least 99.7% identical to SEQ ID NO: 1; and
   ii. a heterologous nucleic acid sequence encoding a sialyl-transferase, preferably a sialyl-transferase selected from table 2, even more preferred a heterologous a sialyl-transferase of SEQ ID NO: 3 or SEQ ID NO: 4 or a functional variant thereof which has least 70%, such as 80%, such as 90%, such as 95% to SEQ ID NO: 3 or SEQ ID NO: 4; and
   iii. a biosynthetic pathway for making a sialate sugar nucleotide; and
b) culturing said cell of (i) in a suitable cell culture medium to allow the HMO production and expression of the DNA sequence to produce the protein having the amino acid sequence of SEQ ID NO: 1, or a functional homologue thereof with an amino acid sequence which is more than 70%, such as at least 80% identical, preferably at least 90% identical, more preferably at least 95, most preferably at least 99.7% identical to SEQ ID NO: 1;
c) harvesting the one or more sialylated HMOs produced in step (ii).

According to the invention, the term “culturing” (or “cultivating” or “cultivation”, also termed “fermentation”) relates to the propagation of bacterial expression cells in a controlled bioreactor according to methods known in the industry.

To produce one or more HMOs, the HMO-producing bacteria as described herein are cultivated according to the procedures known in the art in the presence of a suitable carbon source, e.g., glucose, glycerol, lactose, etc., and the produced HMO is harvested from the cultivation media and the microbial biomass formed during the cultivation process. Thereafter, the HMOs are purified according to the procedures known in the art, e.g., such as described in WO2015/188834, WO2017/182965 or WO2017/152918, and the purified HMOs are used as nutraceuticals, pharmaceuticals, or for any other purpose, e.g., for research.

Manufacturing of HMOs is typically accomplished by performing cultivation in larger volumes. The term “manufacturing” and “manufacturing scale” in the meaning of the invention defines a fermentation with a minimum volume of 5 L culture broth. Usually, a “manufacturing scale” process is defined by being capable of processing large volumes of a preparation containing the product of interest and yielding amounts of the protein of interest that meet, e.g., in the case of a therapeutic compound or composition, the demands for clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regard to the suitable cell medium used in the fermentation process, there are no limitations. The culture medium may be semi-defined, i.e. containing complex media compounds (e.g., yeast extract, soy peptone, casamino acids, etc.), or it may be chemically defined, without any complex compounds.

By the term “one or more HMOs” is meant that an HMO production cell may be able to produce a single HMO structure (a first HMO) or multiple HMO structures (a second, a third, etc. HMO). In some embodiments, it may be preferred a genetically modified cell that produces a single HMO, in other preferred embodiments, a genetically modified cell producing multiple HMO structures may be preferred. Non-limiting examples for genetically modified cell s producing single HMO structures are 2′-FL, 3-FL, 3′-SL, 6′-SL or LNT-2 producing cells. Non-limiting examples of genetically modified cells capable of producing multiple HMO structures can be DFL, FSL, LNT LNnT, LNFP I, LNFP II, LNFP III, LNFP IV, LNFP V, pLNnH, pLNH2, LSTa, LSTb, LSTc, DSLNT, F-LSTa and F-LSTb producing cells.

In particular the present invention relates to genetically modified cells producing one or more of the single HMO structures which are selected from the group consisting of 3′-SL and/or 6′-SL producing cells.

The term “harvesting” in the context in the invention relates to collecting the produced HMO(s) following the termination of fermentation. In different embodiments it may include collecting the HMO(s) included in both the biomass (i.e. the genetically modified cell s) and cultivation media, i.e. before/without separation of the fermentation broth from the biomass. In other embodiments the produced HMOs may be collected separately from the biomass and fermentation broth, i.e. after/following the separation of biomass from cultivation media (i.e. fermentation broth). The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (the production cells). It can be done by any suitable methods of the art, e.g., by sonication, boiling, homogenization, enzymatic lysis using lysozyme, or freezing and grinding.

After recovery from fermentation, HMO(s) are available for further processing and purification.

Purification of HMOs produced by fermentation can be done using a suitable procedure described in WO2016/095924, WO2015/188834, WO2017/152918, WO2017/182965, US2019/0119314 (all incorporated by reference).

HMO products of the invention as described preferably 3′-SL and/or 6′-SL.

### Use

The present invention also relates to the use of a host cell as described herein for use in the production of one or more Human Milk Oligosaccharides (HMOs). In particular, the invention relates to the use of a host cell as described herein for the production of a specific HMO, wherein the host cell is selected with the aim of generating a majority of one specific HMO, preferably selected from 3′-SL and 6′-SL, most preferably 3′-SL.

### Sequences

The current application contains a sequence listing in text format and electronical format which are hereby incorporated by reference.

An overview of the sequences is provided here, if there is discrepancy between the listed sequence and the public reference, the sequence in the sequence list prevails.

TABLE 4
Overview of the sequences

| SEQ ID | | Public reference/ |
|---|---|---|
| NO | Description | origin sequence |
| 1 | Amino acid sequence of MFS transporter Fred | WP_087817556.1 |
| 2 | codon optimized nucleic acid sequence encoding Fred | |
| 3 | Amino acid sequence of α-2,3-sialyl-transferase Nst | AAC44541.1 |
| | Codon optimized sequence encoding Nst | |
| 4 | Amino acid sequence of α-2,6-sialyl-transferase Pd2, ST6 | BAA25316.1 |
| | Codon optimized sequence encoding Pd2, ST6 | |
| 5 | PglpF - promoter | WO2019123324 |
| 6 | PglpF_SD4 -promoter | WO2019123324 |
| 7 | PglpF_SD7 - promoter | WO2019123324 |
| 8 | Plac - promoter | WO2019123324 |
| 9 | Nucleic acid sequence encoding the 2,3-sialyltransferase, NST | — |
| 10 | Nucleic acid sequence encoding the 2,6-sialyltransferase, | — |
| | Pd2, ST6 | |
| 11 | Amino acid sequence of Sialic acid synthase, NeuB | AAK91726.1 |
| 12 | Nucleic acid sequence encoding sialic acid synthase, NeuB | — |
| 13 | Amino acid sequence of N-acetylglucosamine-6- | AAK91727.1 |
| | phosphate 2-epimerase, NeuC | |
| 14 | Nucleic acid sequence encoding N-acetylglucosamine-6- | — |
| | phosphate 2-epimerase, NeuC | |
| 15 | Amino acid sequence of CMP-Neu5Ac synthetase, NeuA | AAK91728.1 |
| 16 | nucleic acid sequence encoding CMP-Neu5Ac synthetase, | — |
| | neuA | |
| 17 | nucleic acid sequence encoding neuCBA gene cluster | — |
| 18 | Amino acid sequence of carboxylating nicotinate- | NP_414651.1 |
| | nucleotide diphosphorylase, nadC | |
| 19 | Nucleic acid sequence encoding carboxylating nicotinate- | — |
| | nucleotide diphosphorylase, nadC | |
| 20 | Oligo O48, galK.for | — |
| 21 | Oligo O49, galK.rev | — |
| 22 | Oligo O40 Backbone.for | — |
| 23 | Oligo O79, Backbone.rev | — |
| 24 | Oligo O261, PglpF.for | — |
| 25 | Oligo O262, PglpF.rev | — |
| 26 | Oligo O459, PglpF_SD4 | — |
| 27 | Oligo O462, PglpF_SD7 | — |
| 28 | Oligo KABY733, fred.for | — |
| 29 | Oligo KABY 734, fred.rev | — |
| 30 | Oligo O68, Plac.for | — |
| 31 | Oligo O113, Plac.rev | — |
| 32 | Oligo KABY721, YberC0001_9420.for | — |
| 33 | Oligo KABY722, YberC0001_9420.rev | — |

Where sequence discrepancies arise between a GenBank ID and a SEQ ID NO, the SEQ ID NO is considered to be the correct sequence.

### General

It should be understood that any feature and/or aspect discussed above in connections with the described invention apply by analogy to the methods described herein.

The following figures and examples are provided below to illustrate the present invention. They are intended to be illustrative and are not to be construed as limiting in any way.

### Items

1. A genetically modified cell capable of producing one or more Human Milk Oligosaccharides (HMO(s)) comprising a sialyl moiety (sialylated HMO), wherein said genetically modified cell comprises,
   (i) a heterologous nucleic acid sequence encoding a major facilitator superfamily (MFS) polypeptide of SEQ ID NO: 1, or a functional homologue thereof with an amino acid sequence which is at least 70% identical to SEQ ID NO: 1, and
   (ii) a heterologous nucleic acid sequence encoding a sialyl-transferase; and
   (iii) a biosynthetic pathway for making a sialate sugar nucleotide.
2. The genetically modified cell according to item 1, wherein the one or more produced HMO(s) is/are selected from the group consisting of 3′-SL, 6′-SL, FSL, DS-LNT, LSTc, LSTa and LSTb.
3. The genetically modified cell according to item 2, wherein the HMO produced by said cell is selected from the group consisting of 3′-SL, 6′-SL and LSTa.
4. The genetically modified cell according to item 2 or 3, wherein the HMO is 3′-SL or 6′SL.
5. The genetically modified cell according to any one of the preceding items, wherein said functional homologue is at least 94.6% identical to SEQ ID NO: 1.
6. The genetically modified cell according to any one of the preceding items, wherein expression of said MFS polypeptide of SEQ ID NO: 1 or a functional homologue thereof leads to increased export of said HMO as compared to a cell without the MFS polypeptide.
7. The genetically modified cell according to item 6, wherein at least 80% of the sialylated HMO is exported out of the genetically modified cell.
8. The genetically modified cell according to any one of the preceding items, wherein the sialyl-transferase is selected from the group consisting of α-2,3-sialyltransferases and α-2,6-sialyltransferases, such as those presented in table 2.
9. The genetically modified cell according to any one of the preceding items, wherein the α-2,3-sialyl-transferase is selected from Nst (SEQ ID NO: 3) or Pd2 (SEQ ID NO: 4), and/or a functional homologue thereof, having an amino acid sequence which is at least 70% identical to SEQ ID NO: 3 or SEQ ID NO: 4.
10. The genetically modified cell according to any one of the preceding items, wherein the sialate sugar nucleotide is CMP-Neu5Ac.
11. The genetically modified cell according to item 10, wherein the cell comprises nucleic acid sequences encoding NeuB of SEQ ID NO: 11, NeuC of SEQ ID NO: 13 and NeuA of SEQ ID NO: 15, or a functional homologue thereof having an amino acid sequence that is at least 80% identical to SEQ ID NO: 11, 13 or 15, respectively
12. The genetically modified cell according to item 10 or 11, wherein the cell comprises a heterologous nucleic acid sequence of SEQ ID NO: 17 encoding a neuBCA cluster, or a functional homologue thereof having at least 80% sequence identity to SEQ ID NO: 17.
13. The genetically modified cell according to any one of the preceding items, wherein the cell further comprises a nucleic acid sequence comprising a regulatory element for the regulation of the expression of the heterologous nucleic acid sequence(s).
14. The genetically modified cell according to item 13, wherein the regulatory element regulates the expression of the MFS polypeptide of SEQ ID NO: 1, ora functional homologue thereof with an amino acid sequence which is at least 70% identical to SEQ ID NO: 1.
15. The genetically modified cell according to item 13 or 14, wherein the regulatory element is selected from the group consisting of PglpF (SEQ ID NO:5), PglpF_SD4 (SEQ ID NO: 6) and PglpF_SD7 (SEQ ID NO:7).
16. The genetically modified cell according to any one of the preceding items, wherein the genetically modified cell is a microbial cell.
17. The genetically modified cell according to item 16, wherein the microbial cell is a bacterium or a fungus.
18. The genetically modified cell according to item 17, wherein said fungus is selected from a yeast cell of the genera Komagataella, Kluyveromyces, Yarrowia, Pichia, Saccaromyces, Schizosaccharomyces or Hansenula or from a filamentous fungous of the genera Aspargillus, Fusarium or Thricoderma.
19. The genetically modified cell according to item 17, wherein said bacterium is selected from the group consisting of Escherichia sp., Bacillus sp., lactobacillus sp. and Campylobacter sp
20. The genetically modified cell according to item 17 or 19, wherein the bacterium is Escherichia coli, such as K-12 or B21.
21. A nucleic acid construct comprising
   (i) a nucleic acid sequence encoding an MFS polypeptide according to SEQ ID NO: 1, or a functional homologue thereof, having more than 70% sequence identity to SEQ ID NO: 1, wherein the nucleic acid sequence encoding the MFS polypeptide has at least 70% sequence identity to SEQ ID NO: 2, and
   (ii) a nucleic acid sequence comprising a regulatory element that regulates the expression of any one or more of the nucleic acid sequence(s) of point (i).
22. A nucleic acid construct comprising
   (i) a nucleic acid sequence encoding an MFS polypeptide according to SEQ ID NO: 1, or a functional homologue thereof, having more than 70% sequence identity to SEQ ID NO: 1, wherein the nucleic acid sequence encoding the MFS polypeptide has at least 70% sequence identity to SEQ ID NO: 2, and
   (ii) a nucleic acid sequence encoding one or more polypeptide(s) with sialyl-transferase capabilities, and/or
   (iii) a nucleic acid sequence comprising a regulatory element that regulates the expression of any one or more of the nucleic acid sequence(s) of point (i) and/or (ii).
23. The nucleic acid construct according to item 21 or 22, wherein the regulatory element is selected from the group consisting of PglpF (SEQ ID NO:5), PglpF_SD4 (SEQ ID NO: 6) and PglpF_SD7 (SEQ ID NO:7).
24. A method for the biosynthetic production of one or more sialylated Human Milk Oligosaccharides (HMO(s)), the method comprising the steps of:
   (i) providing a genetically modified cell according to any one of items 1-20;
   (ii) culturing the genetically modified cell in a suitable cell culture medium to express said heterologous nucleic acid sequence encoding a major facilitator superfamily (MFS) polypeptide of SEQ ID NO: 1, or a functional homologue thereof having an amino acid sequence which is more than 70% identical to SEQ ID NO: 1 and the heterologous nucleic acid sequence encoding a sialyl-transferase to produce one or more sialylated Human Milk Oligosaccharides (HMOs) and;
   (iii) harvesting one or more sialylated HMOs produced in step (ii).
25. The method according to item 24, wherein at least 80% of the sialylated HMO is present in the supernatant of the cell culture.
26. The method according to item 24 or 25, wherein the sialylated HMO, is harvested from the supernatant of the cell culture.
27. The method according to item 24 to 26, wherein the sialylated HMO produces is selected from 3′-SL, and 6′SL.
28. The method according to any one of item 24 to 27, wherein the culture medium comprises an energy source selected from the group consisting of glucose, sucrose, fructose, xylose and glycerol.
29. The method according to any one of item 24 to 28, wherein lactose is added during the cultivation of the genetically engineered cells as a substrate for the HMO formation.
30. Use of a genetically modified cell according to any one of items 1 to 20, or a nucleic acid construct according to any one of items 21 to 23, for the biosynthetic production of one or more sialylated Human Milk Oligosaccharides (HMOs).
31. Use of a genetically modified cell according to any one of items 1 to 20, or a nucleic acid construct according to any one of items 21 to 23 for the biosynthetic production of an HMO selected from the group consisting of 3′-SL and 6′-SL.

### EXAMPLES

### Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Strains

Strains utilized in the present Examples are described in the following table 5.

TABLE 5

| Strain IDs | Product | Relevant Genotype |
|---|---|---|
| DH1 | — | F⁻ λ⁻ endA1 recA1 relA1 gyrA96 thi-1 glnV44 hsdR17(r_{K}⁻m_{K}⁻) |
| MDO | — | E coli DH1 ΔlacZ ΔlacA, ΔnanKETA, ΔmelA, ΔwcaJ, ΔmdoH |
| | | wcaF::Plac |
| Strain 1 | 3′-SL | MDO, 2x Plac-Δ29nst_op ΔnadC pBS-nadC-neuBCA |
| Strain 2 | 3′-SL | MDO, 2x Plac-Δ29nst ΔnadC PglpF-fred pBS-nadC-neuBCA |
| Strain 3 | 3′-SL | MDO, PglpF-neuB_op, PglpF-neuC_op, PglpF-neuA2, PglpF- |
| | | Δ29nst_op |
| Strain 4 | 3′-SL | MDO, PglpF-neuB_op, PglpF-neuC_op, PglpF-neuA2, PglpF- |
| | | Δ29nst_op, PglpF-fred |
| Strain 5 | 3′-SL | MDO, 2xPlac-D29nst_op, ΔnadC, PglpF-yberC; pBS-nadC- |
| | | neuBCA |

### Cultivation

Unless otherwise noted, E. coli strains were propagated in Basal Minimal medium containing 0.2% glucose at 37° C. with agitation. Agar plates were incubated at 37° C. overnight.

Basal Minimal medium had the following composition: NaOH (1 g/L), KOH (2.5 g/L), KH₂PO₄ (7 g/L), NH₄H₂PO₄ (7 g/L), Citric acid (0.5 g/l), Trace mineral solution (5 mL/L). The trace mineral stock solution contained: ZnSO₄*7H₂O 0.82 g/L, Citric acid 20 g/L, MnSO₄*H₂O 0.98 g/L, FeSO₄*7H₂O 3.925 g/L, CuSO₄*5H₂O 0.2 g/L. The pH of the Basal Minimal Medium was adjusted to 7.0 with 5 N NaOH and autoclaved. Before inoculation, the Basal Minimal medium was supplied with 1 mM MgSO_{4,} 4 μg/mL thiamine, 0.5% of a given carbon source (glycerol (Carbosynth)). Thiamine, and antibiotics, were sterilized by filtration. All percentage concentrations for glycerol are expressed as v/v and for glucose as w/v.

### Chemical Competent Cells and Transformations

E. coli was inoculated from LB plates in 5 mL LB containing 0.2% glucose at 37° C. with shaking until OD600 ˜0.4. 2 mL culture was harvested by centrifugation for 25 seconds at 13.000 g. The supernatant was removed, and the cell pellet resuspended in 600 μL cold TB solutions (10 mM PIPES, 15 mM CaCl₂, 250 mM KCl). The cells were incubated on ice for 20 minutes followed by pelleting for 15 seconds at 13.000 g. The supernatant was removed, and the cell pellet resuspended in 100 μL cold TB solution. Transformation of plasmids were done using 100 μL competent cells and 1 to 10 ng plasmid DNA. Cells and DNA were incubated on ice for 20 minutes before heat shocking at 42° C. for 45 seconds. After 2 min incubation on ice 400 μL SOC (20 g/L tryptone, 5 g/L Yeast extract, 0.5 g/L NaCl, 0.186 g/L KCl, 10 mM MgCl2, 10 mM MgSO4 and 20 mM glucose) was added and the cell culture was incubated at 37° C. with shaking for 1 hour before plating on selective plates.

Plasmid were transformed into TOP10 chemical competent cells at conditions recommended by the supplier (ThermoFisher Scientific).

### DNA Techniques

Plasmid DNA from E. coli was isolated using the QIAprep Spin Miniprep kit (Qiagen). Chromosomal DNA from E. coli was isolated using the QIAmp DNA Mini Kit (Qiagen). PCR products were purified using the QIAquick PCR Purification Kit (Qiagen). DreamTaq PCR Master Mix (Thermofisher), Phusion U hot start PCR master mix (Thermofisher), USER Enzym (New England Biolab) were used as recommended by the supplier. Primers were supplied by Eurofins Genomics, Germany. PCR fragments and plasmids were sequenced by Eurofins Genomics. Colony PCR was done using DreamTaq PCR Master Mix in a T100™ Thermal Cycler (Bio-Rad).

The heterologous proteins expressed in the genetically modified HMO producing cells of this invention is described in table 6, the promoter elements which was used in the below exemplifications of the invention is described in table 7, and the oligos used for amplification of plasmid backbones, promoter, elements, and fred is described in table 8.

TABLE 6
The heterologous proteins which can be expressed
in the genetically modified HMO producing cells

| | | Protein | |
|---|---|---|---|
| | Origin of | Accession | |
| Gene | Genes | Number | Protein Function |
| futC | Helicobacter | WP_080473865.1 | α-1,2-fucosyl-transferase |
| | pylori 26695 | | |
| futA | Helicobacter | WP_000487428.1 | α-1,3-fucosyl-transferase |
| | pylori 26695 | | |
| IgtA | Neisseria | WP_002248149.1 | β-1,3-acetylglucosaminyltransferase |
| | meningitidis | | |
| | 053442 | | |
| galTK | Helicobacter | BD182026 | β-1,3-galactosyltransferase |
| | pylori 43504 | | |
| fred* | Yersinia | WP_087817556.1 | MFS transporter |
| | frederiksenii | | |
| yberC0001_9420 | Yersinia | EEQ08298.1 | Major facilitator superfamily MFS_1 |
| | bercovieri | | |
| Δ29nst_op | Neisseria | AAC44541.1 | α-2,3-sialyltransferase |
| | meningitidis | | |
| nadC | Escherichia coli | WP_101348535.1 | quinolinate phosphoribosyl-transferase |
| neuBCA | Campylobacter | WP_002874241.1 | Sialic acid synthase, |
| | jejuni | WP_002874240.1 | N-acetylglucosame-6-phosphate epimerase, |
| | | WP_002874239.1 | CMP-Neu5Ac synthetase |

*the gene name is given to identify the nucleic acid encoding the protein having amino acid sequence of the corresponding GenBank Accession Number for the purposes of the present invention.

TABLE 7
The synthetic regulatory elements utilized for expression of fred
and the sialyl-transferase are identified by the SEQ ID NO's below

| Sequence | | |
|---|---|---|
| name | SEQ ID NO | Description |
| PglpF | 5 | 300-nucleotide DNA expression element |
| PglpF_SD4 | 6 | 300-nucleotide DNA expression element |
| PglpF_SD7 | 7 | 300-nucleotide DNA expression element |
| Plac | 8 | 195-nucleotide DNA expression element |

TABLE 8
Oligos used for amplification of plasmid backbones, promoter,
regulatory elements, and genes of interest, including fred.

| Name | SEQ ID NO | Description |
|---|---|---|
| O40 | 22 | Backbone.for |
| O79 | 23 | Backbone.rev |
| O261 | 24 | PglpF.for |
| O262 | 25 | PglpF.rev |
| O459 | 26 | PglpF_SD4 |
| O462 | 27 | PglpF_SD7 |
| KABY733 | 28 | fred.for |
| KABY734 | 29 | fred.rev |
| O48 | 20 | galK.for |
| O49 | 21 | galK.rev |
| O68 | 30 | Plac.for |
| O113 | 31 | Plac.rev |
| KABY721 | 32 | yberC0001_9420.for |
| KABY722 | 33 | yberC0001_9420.rev |

### Construction of Plasmids

Plasmid backbones containing two I-Scel endonuclease sites, separated by two DNA fragments appropriated for homologous recombination into the E. coli genome and a T1 transcriptional terminator sequence were synthesized. For example, in one plasmid backbone (pUC57::gal) the gal operon (required for homologous recombination in galK), and a T1 transcriptional terminator sequence was synthesized (GeneScript). The DNA sequences used for homologous recombination in the gal operon covered base pairs 3.628.621-3.628.720 and 3.627.572-3.627.671 in sequence Escherichia coli K-12 MG155 complete genome GenBank: ID: CP014225.1. Insertion by homologous recombination would result in a deletion of 949 base pairs of galK and a galK-phenotype. In similar ways backbones based on pUC57 (GeneScript) or an any other appropriated vector containing two I-Scel endonuclease sites, separated by two DNA fragments appropriated for homologous recombination into the E. coli genome and a T1 transcriptional terminator sequence could be synthesized. Standard techniques well-known in the field of molecular biology were used for designing of primers and amplification of specific DNA sequences of the Escherichia coli K-12 DH1 chromosomal DNA. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.).

Chromosomal DNA obtained from E. coli DH1 was used to amplify a 300 bp DNA fragment containing the promoter PglpF using oligos 0261 and 0262, PglpF_SD4 using oligos 0261 and 0459, and PglpF_SD7 using oligos O261 and O462 (Table 8).

A 1.182 bp DNA fragment containing a codon optimized version of the fred gene, SEQ ID NO: 2, originating from Yersinia frederiksenii was synthesized by Genescript (Table 6). The fred gene was amplified using oligos KABY733 and KABY734.

All PCR fragments (plasmid backbones, promoter containing elements and the fred gene) were purified, and plasmid backbones, promoter elements (Plac, PglpF, PglpF_SD4 or PglpF_SD7), and a fred (or other gene of interest, see table 6) containing DNA fragment were assembled. The plasmids were cloned by standard USER cloning. Cloning in any appropriated plasmid could be done using any standard DNA cloning techniques. The plasmids were transformed into TOP10 cells and selected on LB plates containing 100 μg/mL ampicillin (or any appropriated antibiotic) and 0.2% glucose. The constructed plasmids were purified and the promoter sequence and the 5′end of the fred gene was verified by DNA sequencing (MWG Eurofins Genomics). In this way, a genetic cassette containing any promoter of interest linked to the fred (or other gene of interest, see table 6) gene was constructed.

### Construction of Strains

The bacterial strain used, MDO, was constructed from Escherichia coli K-12 DH1. The E. coli K-12 DH1 genotype is: F⁻, λ-, gyrA96, recA1, relA1, endA1, thi-1, hsdR17, supE44. In addition to the E. coli K-12 DH1 genotype MDO has the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Insertion of an expression cassette containing a promoter linked to the fred gene and to a T1 transcriptional terminator sequence in the chromosomal DNA of E. coli K-12 DH1 MDO was performed by Gene Gorging essentially as described by Herring et al. (Herring, C. D., Glasner, J. D. and Blattner, F. R. (2003). Gene (311). 153-163). Briefly, the donor plasmid and the helper plasmid were co-transformed into MDO and selected on LB plates containing 0.2% glucose, ampicillin (100 μg/mL) or kanamycin (50 mg/mL) and chloramphenicol (20 μg/mL). A single colony was inoculated in 1 mL LB containing chloramphenicol (20 μg/mL) and 10 μL of 20% L-arabinose and incubated at 37° C. with shaking for 7 to 8 hours. For integration in the galK loci of E. coli cells were then plated on M9-DOG plates and incubated at 37° C. for 48 hours. Single colonies formed on MM-DOG plates were re-streaked on LB plates containing 0.2% glucose and incubated for 24 hours at 37° C. Colonies that appeared white on MacConkey-galactose agar plates and were sensitive for both ampicillin and chloramphenicol were expected to have lost the donor and the helper plasmid and contain an insertion in the galK loci. Insertions in the galK site was identified by colony PCR using primers O48 (SEQ ID NO: 20) and O49 (SEQ ID NO: 21) and the inserted DNA was verified by sequencing (Eurofins Genomics, Germany).

Insertion of genetic cassettes at other loci in the E. coli chromosomal DNA was done in a similar way using different selection marker genes and different screening methods.

### Deep Well Assay

The deep well assay was performed as originally described to Lv et al (Bioprocess Biosyst Eng (2016) 39:1737-1747) and optimized for the purposes of the current invention.

More specifically, the strains disclosed in the examples were screened in 24 deep well plates using a 4-day protocol. During the first 24 hours, shaker culture cells were grown at 34° C. with 700 rpm shaking, to high densities while in the next 48 hours for 2′-FL, 3-FL and 3′-SL producing cells, and 72 hours for LNT producing cells, cells were transferred to a medium that allowed induction of gene expression and product formation. Specifically, during day 1 fresh inoculums were prepared using a basal minimal medium supplemented with magnesium sulphate, thiamine and glucose. After 24 hours of incubation, cells were transferred to a new basal minimal medium (2 ml) supplemented with magnesium sulphate and thiamine with addition of an initial bolus consisting of 20% glucose solution (1 μl) and 10% lactose solution (0.1 ml) were added, then 50% sucrose solution (0.04 ml) as carbon source was provided to the cells accompanied by the addition of sucrose hydrolase (invertase, 5 μl of a 0.1 g/L solution) so that glucose was provided ata slow rate for growth by cleavage of sucrose by the invertase. After inoculation of the new medium, cells were shaken at 700 rpm at 28° C. for 48 hours. After denaturation and subsequent centrifugation, the supernatants were analysed by HPLC. For the analysis of total samples, the cell lysate prepared by boiling was pelleted by centrifugation for 10 minutes at 4.700 rpm. The HMO concentration in the supernatant was determined by HPLC or HPAC methods.

### 3′-SL Fermentation Process and Associated Measurements

All fermentations were carried out in 200 mL Dasbox bioreactors (Eppendorf, Germany) with a respective starting volume of 100 mL. The medium was a defined minimal culture medium, consisting of 25 g/kg carbon source (glucose), 30 g/kg lactose, MgSO4×7H₂O, KOH, NaOH, NH₄H₂PO₄, KH₂PO₄, trace element solution, citric acid, antifoam and thiamine. The trace metal solution (TMS) contained Mn, Cu, Fe, Zn as sulfate salts and citric acid. Fermentations were started by inoculation with 2% (v/v) of pre-cultures grown in the above-mentioned defined minimal medium. After depletion of the carbon source contained in the batch medium, a sterile feed solution containing glucose, MgSO₄×7H₂O, TMS, antifoam and lactose was fed continuously in a glucose-limited manner, using a predetermined, linear profile.

The pH throughout fermentation was controlled at 6.8 by titration with NH₄OH solution. Aeration was controlled at 1 VVM using air, and dissolved oxygen was kept above 20% of air saturation, controlled by the stirrer rate. Temperature was continuously kept at 34° C.

Throughout the fermentations, samples were taken to determine the concentration of 3′-SL, sialic acid, lactose and other minor by-products using HPLC. Total broth samples were diluted three-fold in deionized water and boiled for 20 minutes, this was followed by centrifugation at 17000 g for 3 minutes, where after the resulting supernatant was analysed by HPLC, which gave the total broth fraction of the produced 3′-SL. The supernatant fraction of the 3′-SL was analysed by HPLC without boiling and was separated from the pellet by centrifugation at 17000 g for 3 minutes.

The bio wet mass (BWM) was determined by pipetting 1 mL of fermentation broth to a 2 mL microcentrifuge tube with a known weight (g), measuring the weight of the broth sample (g), removing the supernatant after centrifugation (17000 g for 3 min) and finally measuring the weight of the pellet. Dividing the weight of the pellet (g) with the weight of the broth (g) and multiplying the result with 1000 gave the BWM in g/kg. All weight measurements were performed with an analytical balance using 3 decimal accuracy.

The supernatant and pellet distribution were calculated by calculating weight of the supernatant (kg) for each sample taken, using the BWM (g/kg) and the accumulated fermentation broth weight (kg). The supernatant weight (kg) was multiplied by the concentration of 3′-SL in the supernatant (g/L) and divided by the weight of 3′-SL in the total broth (g), finally multiplied by 100 to calculate the ratio of 3′-SL in the supernatant (%). The density of the fermentation broth supernatant was 1 g/cm³.

### Example 1—Production of 3′-SL with and without the MFS Transporter, Fred

Engineering of Escherichia coli for 3′-SL Production Expressing the Fred Gene

TABLE 11
Examples of plasmids used for strain construction

| Plasmid | Relevant genotype | Marker gene |
|---|---|---|
| pACBSR | Para-I-SceI-λ Red, p15A ori, cam* | cam |
| pBS-nadC-neuBCA | pBS-nadC-Plac-neuBCA | nadC |
| | [additional genotype | |
| | information as for table 4] | |

The Escherichia coli K-12 (DH1) MDO strain can be manipulated to express heterologous genes of interest. For instance, Strain 1 and Strain 3 are 3′-SL producing strains overexpressing the alpha-2,3-sialyl-transferase, nst, and the CMP-Neu5Ac synthesis enzymes, neuBCA genes. The neuBCA genes can be expressed from the pBS-nadC-neuBCA plasmid (Strain 1) or integrated in the genome (Strain 3). Insertion of an expression cassette containing a promoter element (PglpF) linked to fred in a single copy into the Strain 1 or Strain 3 background resulted in Strain 2 and Strain 4, respectively.

The strains were tested in the deep-well assay as described in as described in the “Materials and methods” section.

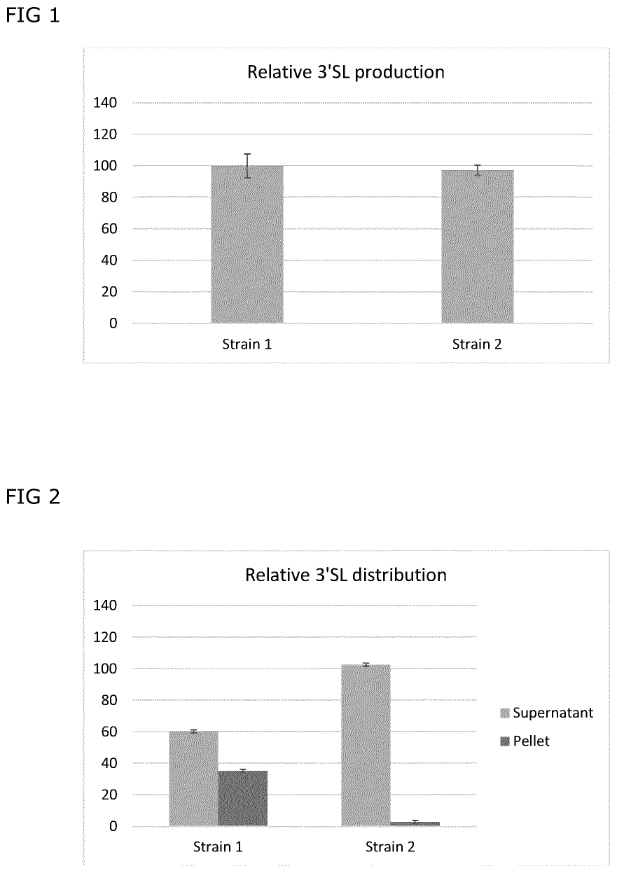

Results from deep-well assays showed that the expression of 3′-SL is not affected by the insertion of the Fred transporter (FIG. 1). The distribution of 3′-SL was however changed significantly with the presence of the Fred transporter, clearly showing that the Fred transporter facilitates transportation of 3′-sl from the cell to the supernatant, leaving only a fraction of the 3′-SL in the pellet, whereas in the absence of the Fred transporter almost 40% 3′-SL remains inside the cell (FIG. 2).

A further evaluation of the strains was performed following fermenting the strains as described in the “Materials and methods” section.

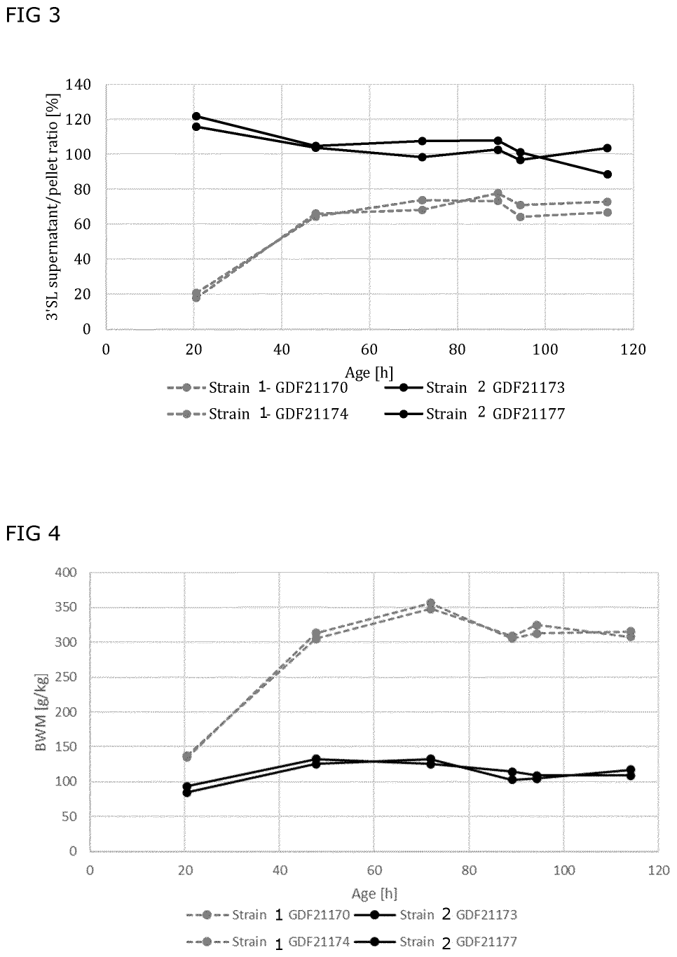

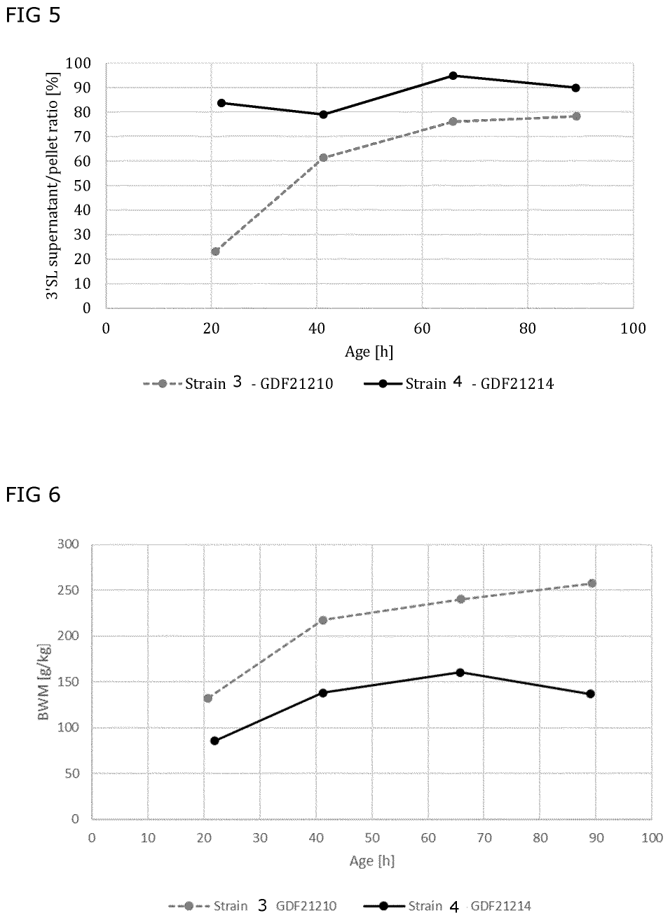

From the fermentation data it can be seen that for strains without the Fred transporter (strain 1 and 3) the 3′-SL is initially high inside the cell and over time it is released to the supernatant, whereas the cells with the Fred transporter (Strain 2 and 4) seem to start with a high level of 3′-SL in the supernatant (FIGS. 3 and 5).

The presence of the Fred transporter also seems to impact the biomass development, in that the same amount of 3′-SL is produced, however when the Fred transporter is present, significantly less biomass is needed to produce the same amount of 3′-SL (FIGS. 4 and 6), which is an advantage both in terms of energy consumption, but also in the down-stream processing for biomass removal.

### Example 2—Production of 3′-SL with and without the MFS Transporter, YberC

Engineering of Escherichia coli for 3′-SL Production Expressing the yberc Gene

The Escherichia coli K-12 (DH1) MDO strain can be manipulated to express heterologous genes of interest as described in Example 1. In the present example, a strain similar to strain 2 was generated except that it expressed a heterologous copy of YberC instead of Fred. Briefly, an expression cassette containing a promoter element (PglpF) linked to a codon optimized yberC gene in a single copy was inserted into strain 1, thereby generating strain 5.

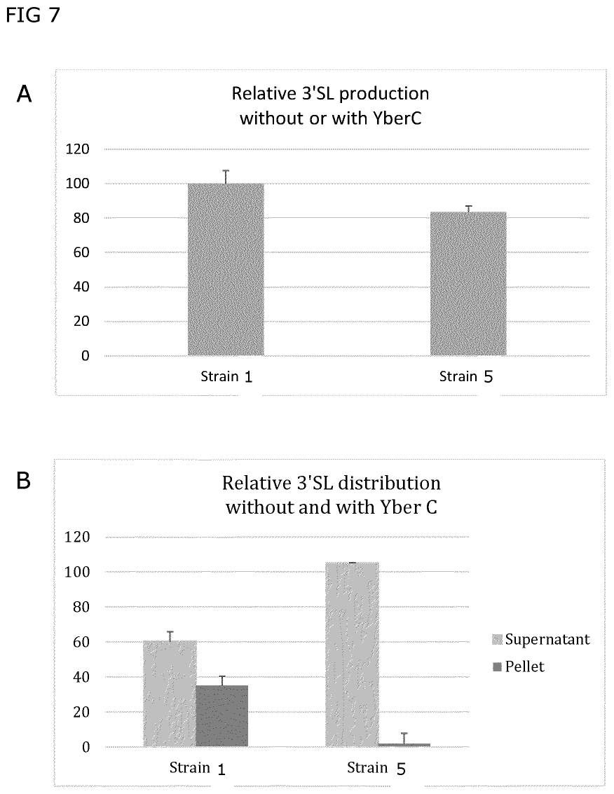

The strains were tested in the deep-well assay as described in as described in the “Materials and methods” section. Results from deep-well assays showed that expression of the yberC gene using PglpF (5) results in approximately 16% reduction in the 3′-SL production compared to the strain without the YberC (strain 1) (FIG. 7A). This indicates that the YberC transporter has a negative effect on the 3′-SL production compared to the Fred transporter.

With respect to the secretion to the supernatant the YberC, like Fred, improves the 3′-SL product distribution by lowering the amount of 3′-SL in the pellet fraction and increasing the amount of 3′-SL in the media (see FIG. 7B).

1. A genetically modified cell capable of producing one or more Human Milk Oligosaccharides (HMO(s)) comprising a sialyl moiety, wherein said genetically modified cell comprises,
(i) a heterologous nucleic acid sequence encoding a major facilitator superfamily (MFS) polypeptide of SEQ ID NO: 1, or a functional homologue thereof with an amino acid sequence which is at least 70% identical to SEQ ID NO: 1, and
(ii) a heterologous nucleic acid sequence encoding a sialyl-transferase; and
(iii) a biosynthetic pathway for making a sialate sugar nucleotide.

2. The genetically modified cell according to claim 1, wherein the one or more produced HMO(s) is/are selected from the group consisting of 3′-SL, 6′-SL, FSL, DS-LNT, LSTc, LSTa and LSTb.

3. The genetically modified cell according to claim 1, wherein expression of said MFS polypeptide of SEQ ID NO: 1 or a functional homologue thereof leads to increased export of said HMO as compared to a cell without the MFS polypeptide.

4. The genetically modified cell according to claim 1, wherein the sialyl-transferase is selected from the group consisting of α-2,3-sialyltransferases and α-2,6-sialyltransferases, such as those presented in table 2.

5. The genetically modified cell according to claim 1, wherein the α-2,3-sialyl-transferase is selected from Nst (SEQ ID NO: 3) or Pd2 (SEQ ID NO: 4), and/or a functional homologue thereof, having an amino acid sequence which is at least 70% identical to SEQ ID NO: 3 or SEQ ID NO: 4.

6. The genetically modified cell according to claim 1, wherein the sialate sugar nucleotide is CMP-Neu5Ac.

7. The genetically modified cell according to claim 1 wherein the biosynthetic pathway for making a sialate sugar nucleotide is encoded by the neuBCA gene cluster of SEQ ID NO: 17 or one or more heterologous genes NeuB of SEQ ID NO: 12 and NeuC of SEQ ID NO: 14 and NeuA of SEQ ID NO: 16 or a functional homologue thereof having a nucleic acid sequence that is at least 80% identical SEQ ID NO: 17, 12, 14 or 16.

8. The genetically modified cell according to claim 1, wherein the cell further comprises a nucleic acid sequence comprising a regulatory element for the regulation of the expression of the heterologous nucleic acid sequence(s).

9. The genetically modified cell according to claim 8, wherein the regulatory element regulates the expression of the MFS polypeptide of SEQ ID NO: 1, or a functional homologue thereof with an amino acid sequence which is at least 70% identical to SEQ ID NO: 1.

10. The genetically modified cell according to claim 8, wherein the regulatory element is selected from the group consisting of PglpF (SEQ ID NO:5), PglpF_SD4 (SEQ ID NO: 6) and PglpF_SD7 (SEQ ID NO:7).

11. The genetically modified cell according to claim 1, wherein the genetically modified cell is a microbial cell, such as Escherichia coli.

12. A nucleic acid construct comprising
(i) a nucleic acid sequence encoding an MFS polypeptide according to SEQ ID NO: 1, or a functional homologue thereof, having more than 70% sequence identity to SEQ ID NO: 1, wherein the nucleic acid sequence encoding the MFS polypeptide has at least 70% sequence identity to SEQ ID NO: 2, and
(ii) a nucleic acid sequence comprising a regulatory element that regulates the expression of any one or more of the nucleic acid sequence of point (i).

13. The nucleic acid construct according to claim 12, wherein the regulatory element is selected from the group consisting of PglpF (SEQ ID NO:5), PglpF_SD4 (SEQ ID NO: 6) and PglpF_SD7 (SEQ ID NO:7).

14. A method for the biosynthetic production of one or more sialylated Human Milk Oligosaccharides (HMO(s)), the method comprising the steps of:
(i) providing a genetically modified cell according to claim 1;
(ii) culturing the genetically modified cell in a suitable cell culture medium to express said heterologous nucleic acid sequence encoding a major facilitator superfamily (MFS) polypeptide of SEQ ID NO: 1, or a functional homologue thereof having an amino acid sequence which is more than 70% identical to SEQ ID NO: 1 and the heterologous nucleic acid sequence encoding a sialyl-transferase to produce one or more sialylated Human Milk Oligosaccharides (HMOs) and;
(iii) harvesting one or more sialylated HMOs produced in step (ii).

15. Use of a genetically modified cell according to claim 1, or a nucleic acid construct, for the biosynthetic production of one or more sialylated Human Milk Oligosaccharides (HMOs).

16. The use of a genetically modified cell according to claims 15, wherein the sialylated HMO is selected from the group consisting of 3′-SL and 6′-SL.
